(19) United States
(12) Patent Application Publication
(10) Pub. No.: US 2006/0183127 A1
(43) Pub. Date: Aug. 17, 2006

(54) Use of silver gene for the authenication of the racial origin of animal populations and of the derivative products thereof

(76) Inventors: Ahmad Oulmouden, Panazol (FR); Raymond Julien, Limoges (FR); Marie-Pierre Laforet, Panazol (FR); Hubert Leveziel, Limoges (FR)

Correspondence Address: YOUNG & THOMPSON, 745 SOUTH 23RD STREET, 2ND FLOOR, ARLINGTON, VA, 22202, US

(21) Appl. No.: 10/565,646

(22) PCT Filed: Jul. 22, 2004
(86) PCT No.: PCT/FR04/01952

§ 371 Date: Mar. 24, 2006

(30) Foreign Application Priority Data
Jul. 25, 2003 (FR) 03/09161

(51) Int. Cl. C12Q 1/68

(52) U.S. Cl. 435/6

(57) ABSTRACT

The invention relates to the use of nucleotide sequences corresponding to the sliver gene, also known as the SI gene, and to the different allelic forms thereof, or corresponding to fragments of the gene and of the different allelic forms of same, in order to perform a method for the identification of different populations or races of ruminant mammals, such as cattle, sheep or goats.

A subject of the invention is the use of nucleotide sequences corresponding to the SILVER gene, also designated SI gene, and to the different allelic forms of this gene, or corresponding to fragments of this gene or of its different allelic forms, for the implementation of a method for the identification of different populations or breeds of ruminant mammals such as cattle, sheep and goats.

Since the recent food crises in Europe, the consumer wants to be informed about the origin of the product: the concept of breed has become determinant for quality cattle networks, as it is associated with a stock-farming system increasing the value of the land.

The French national cattle herd includes more than 20 million head of cattle distributed among some forty breeds which generally have a well identified regional origin and a typical coat colour, a legacy of the work of the first selective breeders in the 18th and 19th centuries. In the case of quality beef production, the breed of origin of the product is a determinant component of the specifications which the farmer wants to guarantee to the consumer. The name of the breed becomes a trademark highlighted by the distributor (for example the Blason Prestige label rouge for the Limousin breed, the Boeuf Gascon label rouge, or the Maine-Anjou AOC (controlled designation of origin) in the case of the Rouge des Prés breed). Thus, the “quality labels” of the products make clear the origin, the specifications, the type of feed and the breed, but also present a photograph of the animal on the hoof, showing the informative value of its appearance, its colour being a major factor. Moreover, the colouring of the coat has played a determinant role in the history of cattle breeds: whereas a cattle breed exhibits variability in performances and genome, it is precisely described and fixed as regards its colour, which therefore becomes a characteristic element in respect of the breed to which an animal belongs, and the products derived from it.

The present invention results from the identification by the Inventors of a major gene in the colouring of cattle, namely the SILVER gene, and the fact that the polymorphism of this gene in animals is a characteristic of the breed to which an animal belongs. Two alleles of this gene have been identified. The first, the si allele, peculiar to the Charolais breed causes the creamy white colouring of its coat. The second, si₁, affects the same region as the Charolais allele and characterizes another cattle breed, namely the Simmental breed.

Although anonymous molecular markers such as microsatellites make it possible to assign an animal to a breed with a high probability, they do not however make it possible to guarantee that all the criteria defining the breed will be met and prove very inefficient in the case of samples originating from cross-bred animals. On the other hand, the invention makes it possible to guarantee that the marketed products meet the criteria attached to the breed, and in particular its standard, attested to by the colour of the coat. Moreover, in order to propose an individual traceability molecular test, it would be necessary to genotype all the animals slaughtered, in order to establish correspondence between the typing of a sample of meat and the genotype of a given animal. For breed traceability, a single typing is to be carried out in order to be compared with the reference genotype characteristic of the breed. The breed traceability test of the present invention is simpler to implement than an individual traceability test. In this context, the invention now makes it possible to give selective breeders of the Charolais breed a novel means of guaranteeing the breed traceability of their animals and makes it possible, by the same means, for any economic operator, to verify the reliability of his supplier within the framework of his contractual agreements, independently of an official product-certification mechanism.

A subject of the invention is the use of nucleotide sequences corresponding to the SILVER gene, also designated SI gene, and to the different allelic forms of this gene, or corresponding to fragments of this gene or of its different allelic forms, for the implementation of a method for the identification of different populations or breeds of ruminant mammals such as cattle, sheep, and goats.

A more particular subject of the invention is the abovementioned use of nucleotide sequences corresponding to the bovine SILVER gene represented by SEQ ID NO: 1, coding for the bovine SI protein represented by SEQ ID NO: 2, and to the different allelic forms of this gene, or corresponding to fragments of this gene or of its different allelic forms, for the implementation of a method for the identification of different cattle populations or breeds, or of different cattle herds each including several cattle populations or breeds.

The invention relates more particularly to the use of nucleotide sequences as defined above, for the implementation of a method making it possible to check, or even to certify, that an animal belongs to a particular cattle population or breed, or to a particular cattle herd, or, on the other hand, making it possible to certify the exclusion of this animal from this population or breed, or from this particular herd.

A more particular subject of the invention is the use of nucleotide sequences as defined above, characterized in that cattle populations or breeds or cattle herds are of French origin.

The invention also relates to the abovementioned use:

of the nucleotide sequence corresponding to the si allelic form represented by SEQ ID NO: 3, coding for the bovine si protein represented by SEQ ID NO: 4, or corresponding to fragments of this allelic form, said allelic form comprising the G93A mutation with respect to the SI gene, for the implementation of a method making it possible to check, or even to certify, that an animal belongs to the Charolais breed,

of the nucleotide sequence corresponding to the si₁ allelic form, represented by SEQ ID NO: 5, coding for the bovine si₁ protein represented by SEQ ID NO: 6, or corresponding to fragments of this allelic form, said allelic form comprising a deletion of the three nucleotides TTC situated in positions 82, 83 and 84 with respect to the SI gene, for the implementation of a method making it possible to check, or even to certify, that an animal belongs to the Simmental breed,

of the nucleotide sequence corresponding to the bovine SI gene represented by SEQ ID NO: 1, or to fragments of this gene, for the implementation of a method making it possible to certify the exclusion of an animal from the Charolais breed.

The invention also relates to the abovementioned use of fragments of the nucleotide sequences corresponding to the allelic forms, SI, si, and si₁, represented by SEQ ID NO: 1, 3, and 5 respectively, said fragments being chosen from those of approximately 10 to 300 nucleotides containing the nucleotides situated in positions 82 to 93 of said sequences.

The invention relates more particularly to the abovementioned use of fragments of the nucleotide sequences corresponding to the allelic forms SI, si, and si₁, said fragments being chosen from those of 294 nucleotides delimited by the nucleotides situated in positions 9 and 302 of the sequences SEQ ID NO: 1 and 3, these fragments being represented by the sequences SEQ ID NO: 7, and SEQ ID NO: 8 respectively, and the fragment of 291 nucleotides delimited by the nucleotides situated in positions 9 and 299 of the sequence SEQ ID NO: 5, this fragment being represented by the sequence SEQ ID NO: 9.

A subject of the invention is also the use of nucleotide primers making it possible to amplify the number of copies of the SILV gene, or of the different allelic forms of this gene, or of the fragments of this gene or of its different allelic forms, as defined above, for the implementation of a method for the identification of different populations or breeds of ruminant mammals such as cattle, sheep and goats.

The invention relates more particularly to the use of the abovementioned nucleotide primers, in the form of 5′-3′ primer pairs, these pairs being such that:

the 5′ primer is chosen from the SIL10 primer represented by the following sequence SEQ ID NO: 10:

5′ GTTGCTGGAAGGAAGAACAGGATGGATCTG 3′

or any sequence derived from this sequence SEQ ID NO: 10, in particular by suppression and/or substitution and/or addition of one or more nucleotides, said derived sequence being hybridized, like the sequence SEQ ID NO: 10, with all or part of the nucleotide sequence complementary to the nucleotide sequences delimited by the nucleotides situated in positions 9 and 38 of the sequences SEQ ID NO: 1, 3, and 5,

the 3′ primer is chosen from the SIL8 primer represented by the following sequence SEQ ID NO: 11:

5′ CAGTCCCAAGTGCCTGAACACACATGCACC 3′

or any sequence derived from this sequence SEQ ID NO: 11, in particular by suppression and/or substitution and/or addition of one or more nucleotides, said derived sequence being hybridized, like the sequence SEQ ID NO: 11, with all or part of the nucleotide sequences delimited by the nucleotides situated in positions 276 and 302 of the sequences SEQ ID NO: 1, 3, and by the nucleotides situated in positions 273 and 299, of the sequence SEQ ID NO: 5.

The invention also relates to the nucleotide sequence characterized in that it corresponds to the bovine SI gene represented by SEQ ID NO:1, or to the following fragments of the SI gene:

any fragment chosen from those of approximately 10 to 300 nucleotides containing the nucleotides situated in positions 82 to 93 of the sequence SEQ ID NO: 1,

the fragment SEQ ID NO: 7 of 294 nucleotides delimited by the nucleotides situated in positions 9 and 302 of the sequence SEQ ID NO: 1.

A subject of the invention is also the nucleotide sequence characterized in that it corresponds to the bovine si gene represented by SEQ ID NO: 3, or to the following fragments of the si gene:

any fragment chosen from those of approximately 10 to 300 nucleotides containing the nucleotides situated in positions 82 to 93 of the sequence SEQ ID NO: 3,

the fragment SEQ ID NO: 8 of 294 nucleotides delimited by the nucleotides situated in positions 9 and 302 of the sequence SEQ ID NO: 3.

The invention also relates to the nucleotide sequence characterized in that it corresponds to the bovine si₁ gene represented by SEQ ID NO: 5, or to the following fragments of the si₁ gene:

any fragment chosen from those of approximately 10 to 300 nucleotides containing the nucleotides situated in positions 82 to 93 of the sequence SEQ ID NO: 5,

the fragment SEQ ID NO: 9 of 291 nucleotides delimited by the nucleotides situated in positions 9 and 299 of the sequence SEQ ID NO: 5.

A subject of the invention is also the nucleotide sequence characterized in that it comprises:

the SIL 10 nucleotide sequence represented by the following sequence SEQ ID NO: 10:

5′ GTTGCTGGAAGGAAGAACAGGATGGATCTG 3′

or any sequence derived from this sequence SEQ ID NO: 10, in particular by suppression and/or substitution and/or addition of one or more nucleotides, said derived sequence being hybridized, like the sequence SEQ ID NO: 10, with all or part of the nucleotide sequence complementary to the nucleotide sequences delimited by the nucleotides situated in positions 9 and 38 of the sequences SEQ ID NO: 1, 3, and 5,

the SIL8 nucleotide sequence represented by the following sequence SEQ ID NO: 11:

5′ CAGTCCCAAGTGCCTGAACACACATGCACC 3′

or any sequence derived from this sequence SEQ ID NO: 11, in particular by suppression and/or substitution and/or addition of one or more nucleotides, said derived sequence being hybridized, like the sequence SEQ ID NO: 11, with all or part of the nucleotide sequences delimited by the nucleotides situated in positions 276 and 302 of the sequences SEQ ID NO: 1, 3, and by the nucleotides situated in positions 273 and 299, of the sequence SEQ ID NO: 5.

The invention also relates to primer pairs, each of the two primers comprising, independently of one other, approximately 10 to approximately 30 nucleotides, characterized in that they are chosen in such a manner that one of the two sequences of a primer pair is hybridized with a sequence of approximately 10 to approximately 30 nucleotides comprised in the nucleotide sequence complementary to the sequence delimited by the nucleotides situated in positions 1 and approximately 60 of the nucleotide sequences SEQ ID NO: 1, 3, and 5, whilst the other sequence of this same pair is hybridized with a sequence of approximately 10 to approximately 30 nucleotides comprised between the nucleotide situated in position 94 and the last of the nucleotides of the sequences SEQ ID NO: 1, 3, and 5.

A more particular subject of the invention is the primer pairs for gene amplification as defined above, characterized in that:

one of the primers is chosen from the sequences comprising the SIL10 sequence represented by SEQ ID NO: 10, or any sequence derived from the latter, as defined above, said primer being advantageously labelled, in particular in a radioactive or fluorescent manner,

whilst the other primer is chosen from the sequences comprising the SIL8 sequence represented by SEQ ID NO: 11, or any sequence derived from the latter, as defined above.

A subject of the invention is also a method for the identification of populations or breeds of ruminant mammals such as cattle, sheep and goats, said method being carried out starting with a biological sample collected from the animal, in particular starting with sperm, embryo, blood, milk, hairs, carcass or meat, or other products derived from the latter, and making it possible to check, or even to certify, whether or not the animal from which said biological sample was collected belongs to a population or breed of ruminant mammals, this method comprising:

a stage of amplification of the number of copies of the different allelic forms of the SILVER gene, namely of the SI, and/or si, and/or si₁ alleles, and/or of fragments of these allelic forms, specific to a population or breed of specific ruminant mammals, and capable of being present in said biological sample,

a stage of detection of said allelic forms or fragments of the latter.

A more particular subject of the invention is an identification method as defined above, characterized in that the stage of amplification of the number of copies of the different allelic forms of the SILVER gene, or of the fragments of these allelic forms, is carried out using an abovementioned primer pair.

The invention relates more particularly to an identification method as defined above, characterized in that:

the detection of a genotype comprising the si allele in the biological sample studied makes it possible to certify that said sample originates from an animal belonging to the Charolais breed or having at least one ancestor of the Charolais breed,

the detection of a genotype comprising the si₁ allele in the biological sample studied makes it possible to certify that said sample originates from an animal belonging to the Simmental breed or having at least one ancestor of the Simmental breed,
the detection of a genotype comprising the SI allele, makes it possible to certify that said sample does not originate from an animal of the Charolais breed.

A subject of the invention is also a kit for the implementation of a method as defined above, characterized in that it comprises at least one abovementioned primer pair, and if appropriate the reagents necessary for the implementation of the amplification reaction of the number of copies of the different allelic forms of the SILVER gene.

The invention is further illustrated using the detailed description which follows of the detection of the bovine SILVER gene, and its si and si₁ allelic forms.

### KEY TO THE FIGURES

FIG. 1: Transcript of the bovine SILVER gene. The 5′ UTR region is 29 bp. The 3′ UTR region has a size of 107 bp followed by a polyA tail. The polyadenylation site is underlined. The bovine Pmel17 protein is formed by 649 amino acids. The peptide signal is formed by the first 24 amino acids. The Charolais si allele leads to the substitution of a G glycine (G) in position 22 by an arginine (R).

FIG. 2: Genomic structure of the coding region of the bovine SILVER gene. The SIL10 and SIL9 primers which have made it possible to amplify it from the genomic DNA are indicated. The different exons are in bold type. The exon coordinates at the level of this DNA fragment are summarized in Table 2. The GT donor sites and the AG acceptor splice sites are in italics. The Charolais mutation (substitution of the guanine by adenine) is in position 96 (first exon).

FIG. 3: Comparison of the nucleotide sequences of the transcripts of the Charolais SILVER gene and of the RPE1 transcript. The differences between the two sequences are indicated in bold type. These are the absence of 499 bp from the 5′ region and also modifications (A1151C), (CAG1458), (G1461A) and (C1864A). The Charolais mutation (G93A) is in position 93 (in bold type and italics). The translation start codon ATG is in position 30 (in italics) and the stop codon (TGA) is in position 1977 (in italics).

FIG. 4: Comparison of peptide sequences deduced from the transcript of the Charolais SILVER gene and that deduced from the RPE1 transcript. The differences between the two sequences are in bold type. These are the absence of the first 157 amino acids and also modifications Q4677, G478S, and A612E. The modification (in bold type and italics) which characterizes the Charolais breed is in position 22 (G22R).

I) Material and Methods

Extraction of Total RNAs from Bovine Skin Samples

Extraction is carried out starting with a 2 cm² skin sample taken from an individual bovine as rapidly as possible after slaughter. The skin is shaved with a scalpel blade and its adipose layer is removed. The sample is then cut into small pieces. The extraction of the RNAs is carried out using the “RNeasy Maxi Kit” (Qiagen; Reference: 75162) according to the supplier's recommendations. The total RNAs thus prepared are used for the synthesis of the complementary DNA (cDNA). They are also used in the RACE-PCR technique in order to make available the 5′ and 3′ UTRs regions (untranslated regions) of the transcript of the SILVER gene.

Synthesis of the cDNAs

5 μg of the total RNAs of the Charolais and Salers breeds were retrotranscribed using SuperScriptII reverse transcriptase (Invitrogen; reference 18064-014), according to the supplier's recommendations. The complementary DNAs thus obtained are used as a matrix in order to obtain in particular the coding part of the SILVER gene.

Obtaining the 5′ and 3′ Ends of Complementary DNA

The SMART RACE cDNA Amplification Kit (Clontech; reference K1811-1) was used in order to obtain complementary DNAs ligated to 5′ and 3′ adapters. 5 μg of total RNAs were used according to the supplier's recommendations (Clontech).

Purification of Genomic DNA from Blood Samples

5 ml of sang collected in EDTA, transferred into a 50 ml tube (Nalgène) are diluted with 4 volumes of TE 20:5 (20 mM Tris: 5 mM ETDA). The sample is then incubated for 15 minutes on ice for lysis of the red blood cells to take place. After centrifugation at 4000 rpm for 20 minutes at 4° C. the supernatant is slowly eliminated. The cells are then resuspended in 15 ml of TE 20:5, followed by centrifugation for 15 minutes at 4000 rpm and at 4° C. Two washing procedures are carried out (until the pellet is translucent). The pellet is then taken up in ½ of the starting volume of blood with TE 20:5, to which SDS (1% final) and proteinase K (200 μg/ml final) are added. The mixture is incubated under stirring (300 rpm) overnight at 37° C. The DNA is then precipitated by adding ⅓ of the volume to 7.5 M ammonium acetate and 2 volumes of cold absolute ethanol. The pellet is then washed with 70% ethanol, dried, and taken up (100 ng/μl) in water and kept at 4° C.

Amplifications by the PCR Technique

Amplifications of DNA fragments invariably comprise a denaturation phase, a hybridization phase and an extension phase. This cycle is repeated 35 times and it is followed by an extension stage of 7 minutes. The duration of each of the phases as well as the primers used are specified for each amplification.

Purification of DNA Fragments after Separation on Gel

This elution technique is used in cases where it proves necessary to separate a fragment of interest from the contaminant DNA, such as for example for the preparation of an insert before cloning. For this purpose, the “QiaQuick Gel Extraction kit” (Qiagen, reference 28706) was used according to the manufacturer's recommendations.

Cloning Technique

In order to clone the DNA fragments, the inventors used two types of commercial vectors (Invitrogen). The pCR 2.1-TOPO vector (reference K4500-01) for the cloning of DNA fragments smaller than 2 kbp and the TOPO XL vector (reference K4750-20) for the cloning of fragments larger than 2 kbp. The cloning is carried out according to the supplier's recommendations.

Sequencing of DNA Fragments

The sequences of cloned DNA fragments are determined according to the principle of the Sanger technique using an automatic sequencer (ABI Prism 310 Genetic Analyser, Perkin Elmer).

The sequencing reactions are carried out using a thermostable enzyme. They require approximately 200 to 400 μg of plasmid matrix, a commercial reaction mixture (Terminator Ready Mix, PRISM Ready Reaction Ampli Taq FS, Perkin Elmer) containing the dNTPs, ddNTPs, MgCl₂ and DNA polymerase (Taq FS) as well as 15 pmol of primer. Twenty-five cycles are carried out in a final volume of 20 μL: denaturation at 96° C. for 10 seconds, hybridization of the primer at 55° C. for 5 seconds and extension at 60° C. for 4 minutes. The neosynthesized DNA is precipitated by 80 μL of 75% isopropanol, washed with 250 μL of 75% isopropanol in order to eliminate the unused reagents which could disturb the migration, dried then taken up in 20 μL of TSR before being denatured by heat treatment.

II) Results

Experimental Data

Isolation of the 5′ and 3′ UTR Regions of the SILVER Gene of the Charolais Cattle Breed

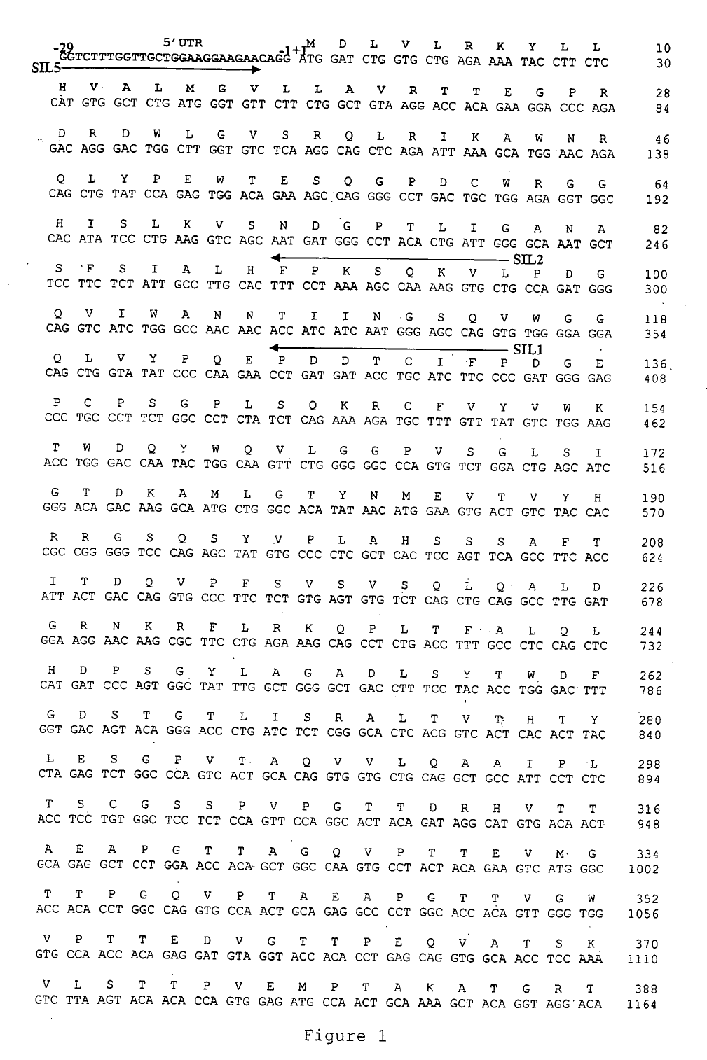

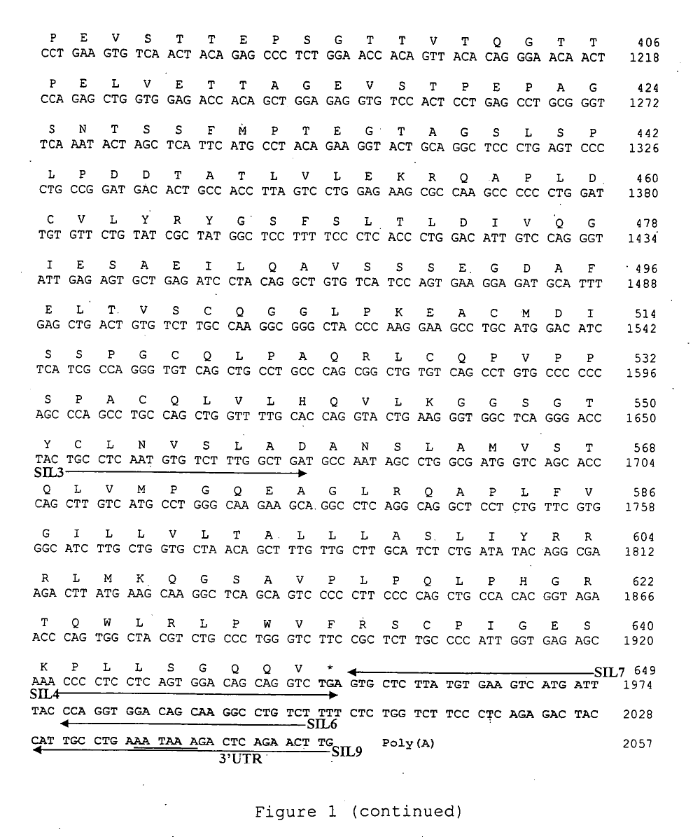

The 5′ and 3′ UTR regions (untranslated transcript regions) of the SILVER gene of the Charolais breed were obtained using primer pairs (FIG. 1) (SIL1/UPM and SIL2/NUP) and (SIL3/UPM and SIL4/NUP) respectively. These primers were deduced from preserved regions of the human (ACC. AK092881) and murine (ACC. AK012808) SILVER genes available from the databases. In the two cases a so-called nested PCR is necessary in order to obtain an amplification product. 1.5 μl of cDNA coupled with the adapters were used as matrix. We used Abgene polymerase (Reference AM-266-615) according to the supplier's recommendations. The PCR conditions are the following: 94° C., 2 minutes, (94° C. 30 seconds, 61° C. 30 seconds, 72° C., 1 min)×35 cycles, 72° C. 7 minutes.

Obtaining the 5′ UTR Region

After a first amplification with the primer pair SIL1/UPM (UPM:10× Universal Primer A Mix; Clontech), a second PCR with the primer pair SIL2/NUP (NUP: Nested Universal Primer A; Clontech) was carried out with 1.5 μl of the first PCR under the same amplification conditions. The amplificate obtained is ligated in the pCR 2.1-TOPO vector and sequenced. The sequence obtained comprises (FIG. 1) the 5′ UTR region of 29 bp and 236 bp of the coding region of the SILVER gene.

Obtaining the 3′ UTR Region

The same procedure was used in order to obtain the 3′ UTR region of the gene by using the primer pairs SIL3/UPM and SIL4/NUP in the first and second PCR respectively. The amplificate obtained was also ligated in the pCR 2.1-TOPO vector and sequenced. The sequence obtained comprises the last 27 base pairs of the coding region (FIG. 1) followed by the 3′ UTR region of 107 bp and ends in a poly(A) tail.

Isolation of the Whole of the Coding Part of the SILVER Gene

Starting with information on the sequences of the 5′ and 3′ UTR regions (FIG. 1) we synthesized three primers SIL5 (sense), SIL6 and SIL17 (anti-sense). These primers were used in a first PCR (SIL5/SIL6) and second PCR (SIL5/SIL7) in order to amplify the whole of the coding part of the SILVER gene from the cDNA of the Charolais and Salers breeds. 4 μl of each of the amplificates (Charolais and Salers) were cloned separately in the TOPO XL vector (Invitrogen) and sequenced. FIG. 1 shows the whole of the sequence obtained for the Charolais breed.

Discovery of the Charolais Allele

Comparison of the coding sequences of the Charolais and Salers SILVER gene revealed a single difference: the substitution of an adenine in position 64 in the Salers for a guanine (FIG. 1) in the Charolais. We have designated this allele of the SILVER gene: si.

Establishment of the Genomic Structure of the Coding Part of the Silver Gene

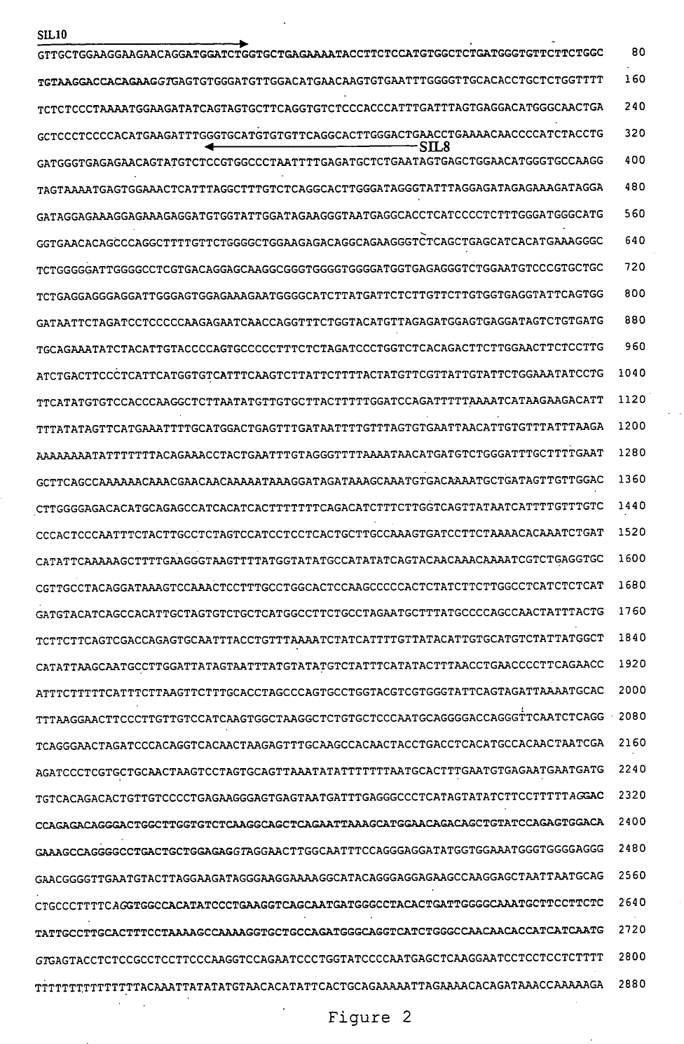

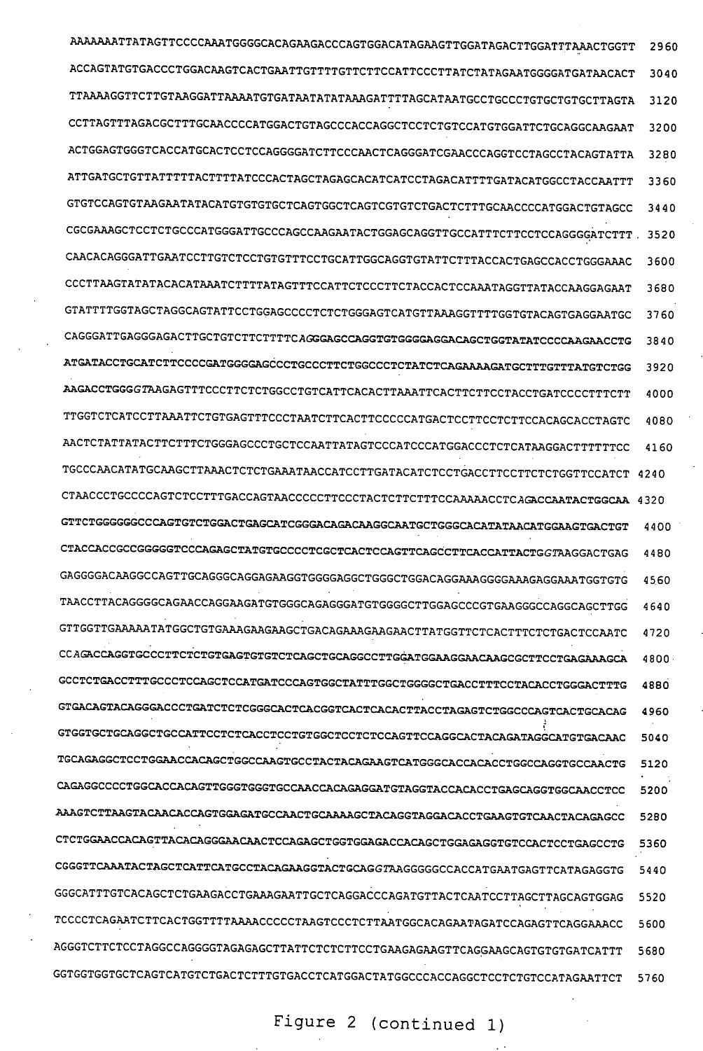

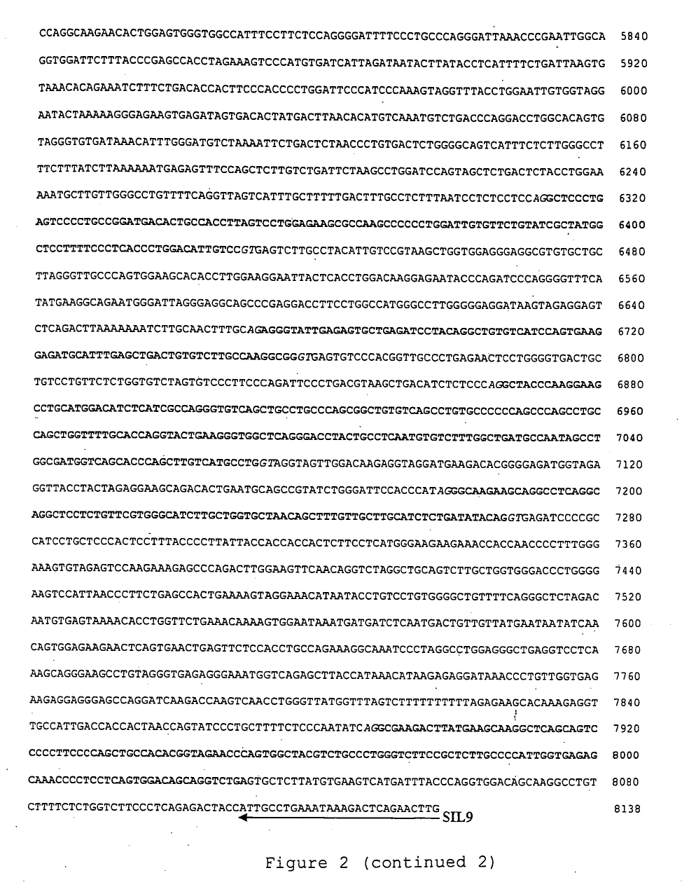

In order to obtain all of the genetic information containing the coding part of the SILVER gene of the Charolais breed, we used the primer pair SIL5/SIL9 (FIG. 1). By using a Taq polymerase (Expand Long Template PCR System, Roche, reference 1 681 834) which makes it possible to amplify DNA fragments up to 20 kbp, we obtained a fragment of Charolais genomic DNA of approximately 8 kbp. The latter was cloned in the TOPO XL vector (Invitrogen) and entirely sequenced. FIG. 2 represents the exon/intron structure of the coding part of the Charolais SILVER gene.

Genotyping of the si Allele

In order to study this region, we synthesized a primer (SIL8) in the first intronic region (FIG. 2). By using the primer pair SIL10/SIL8, a genomic DNA fragment of 294 bp is obtained. The direct sequencing of the amplificate immediately teaches us about the situation of the Charolais mutation in the other breeds. We also analyzed the case of certain cross-breeds one of the parents of which is a Charolais individual. Table 1 below shows the results of si allele genotyping for different cattle breeds.

Table 1
Genotyping of the si and si₁ allele of different cattle breeds. The si allele
is characteristic of all the individuals of the Charolais breed. Five cross-
bred animals one of the parents of which is a Charolais were also
genotyped for the si allele. One of the crossbreeds is si/si₁ heterozygous.
It carries the Charolais si allele and the si₁ allele. The si₁ allele
characterizes individuals of Simmental origin.

| | | | | | | | | Number |
|---|---|---|---|---|---|---|---|---|
| | | | | | | | | of indi- |
| | | Alleles of the SILVER gene | | | | | | viduals |
| Bovine breeds | | si/si | SI/SI | SI/si | si/si₁ | SI/si₁ | si₁/si₁ | tested |
| Charolais | | + | − | − | − | − | − | 41 |
| Limousine | | − | + | − | − | − | − | 11 |
| Blonde d'Aquitaine | | − | + | − | − | − | − | 5 |
| Salers | | − | + | − | − | − | − | 8 |
| Maine Anjou | | − | + | − | − | − | − | 3 |
| Montbéliarde | | − | + | − | − | − | − | 3 |
| Gasconne | | − | + | − | − | − | − | 12 |
| Aubrac | | − | + | − | − | − | − | 10 |
| Parthenaise | | − | + | − | − | − | − | 3 |
| Bazadaise | | − | + | − | − | − | − | 6 |
| Normande | | − | + | − | − | − | − | 10 |
| Blanc bleue | | − | + | − | − | − | − | 6 |
| Prim'Holstein | | − | + | − | − | − | − | 1 |
| Cross-breeds | 4 | − | − | + | − | − | − | 5 |
| Charolais | 1 | − | − | − | + | − | − | |
| Simmental | 2 | − | + | − | − | − | − | 12 |
| | 5 | − | − | − | − | + | − | |
| French | 5 | − | − | − | − | − | + | |

III) Analysis of the Experimental Data

Transcript and Product of the Bovine Silver Gene

The mRNA of the bovine SILVER gene has a size of 2086 bp. The 5′ and 3′ UTR regions have a size of 29 bp and 107 bp respectively (FIG. 1). The open reading frame has a size of 1950 bp and codes a protein of 649 amino acids with a peptide signal of 24 amino acids (FIG. 1).

Genomic Structure of the Coding Part

The coding part of the bovine SILVER gene comprises 11 exons and 10 introns (FIG. 2).

The size and position of the different exons are mentioned in Table 2 hereafter. The donor (GT) and acceptor (AG) splice sites are perfectly preserved.

TABLEAU 2
Positions of the coding exons at the genomic level and at the transcript
level of the bovine SILVER gene. The position of the first nucleotide at
the genomic level corresponds to the first 5′ nucleotide of the
SIL10 primer (FIG. 2). At the level of the transcript the first nucleotide
corresponds to the start of the transcription (FIG. 1).

| Coding | Coordinates at the | | Sizes |
|---|---|---|---|
| exons | genomic level | transcript level | bp |
| Exon 1 | 22—97 | 30—105 | 76 |
| Exon 2 | 2318—2428 | 106—216 | 111 |
| Exon 3 | 2574—2720 | 217—363 | 147 |
| Exon 4 | 3796—3930 | 364—498 | 135 |
| Exon 5 | 4307—4468 | 499—660 | 162 |
| Exon 6 | 4725—5405 | 661—1341 | 681 |
| Exon 7 | 6313—6429 | 1342—1458 | 117 |
| Exon 8 | 6673—6757 | 1459—1543 | 85 |
| Exon 9 | 6867—7072 | 1544—1749 | 206 |
| Exon 10 | 7180—7267 | 1750—1837 | 88 |
| Exon 11 | 7890—8031 | 1838—1979 | 142 |

Breed to which the si Allele Belongs, and the Discovery of a Novel Allele

Analysis of the genotyping data (Table 1) demonstrates that the si allele in the homozygous state is encountered only in the case of individuals belonging to the Charolais breed. First-generation cross-bred individuals one of the parents of which is Charolais are also identifiable by their heterozygous genotype (si/SI).

One of the crossbreeds (Table 1) which we analyzed has the following genotype: si/si₁. It carries the Charolais allele and a novel si₁ allele. The si₁ allele, is characterized (FIG. 1) by the deletion of three nucleotides T, T, and C in positions 53, 54 and 55 respectively (ΔT53, ΔT54 and ΔC55). The positions are indicated with respect to the first translation start nucleotide (adenine A). This deletion engenders (FIG. 1) the loss of an amino acid leucine in position 18 (AL18). Because of the position of this deletion (adjacent to the Charolais mutation) in addition to the role of the amino acid (leucine) in the effectiveness of the elimination of the peptide signal, this second allele of the SILVER gene must have an effect similar to the Charolais allele: dilution of the colour of the coat. One of the breeds, if not the only one which possesses a dilution effect similar to that of the Charolais is the Simmental breed.

Genotyping of the si₁ Allele

We genotyped 12 individuals belonging to the French Simmental breed. The results obtained are presented in Table 1. The si₁ allele is encountered in the French Simmental breed in the homozygous state (si₁/si₁) and in the heterozygous state (si₁/SI). The wild-type allele in the homozygous state (SI/SI) is also encountered.

Homologies with the Data Available in the Databases

1. Human and Murine Data

The bovine Silver protein (also called Pmel17) has 79% and 76% homology with its human (ACC. AK092881) and murine (ACC. AK092881) homologues respectively. It is to be noted that the amino acid glycine (G) which is substituted by an arginine (R) in the case of the Charolais cattle breed (FIG. 1) is preserved in other cattle, humans and mice.

2. Bovine Data

Comparison of the sequence of the transcript of the bovine SILVER gene with the data available in the databases reveals the presence of information on sequences of the bovine SILVER gene. These sequences are of two types: ESTs (ACC:BM106313; AW352955; AW478070; BF604634; BF599555), and also a partial sequence (ACC: M81193) of the transcript of the bovine SILVER gene.

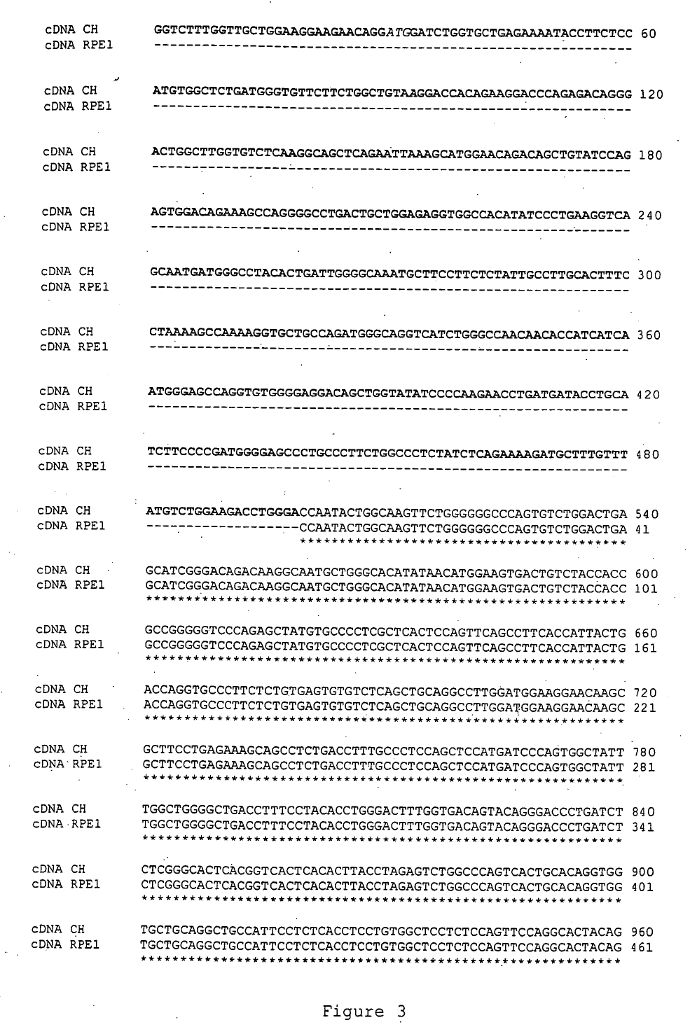

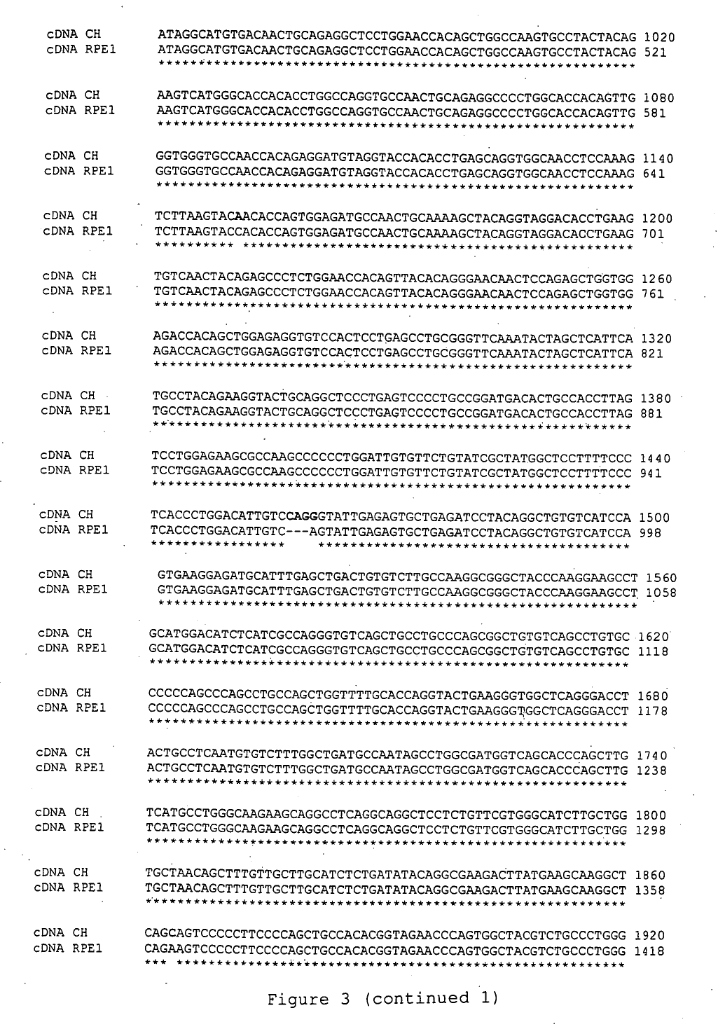

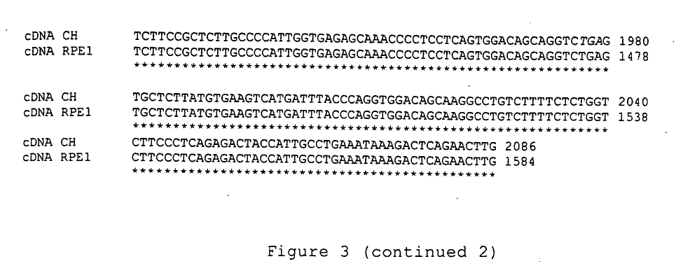

This partial sequence has been published under the name of RPE1 (retinal pigment epithelium) in 1992 by Kim R Y and Wistow G J (The cDNA RPE1 and monoclonal antibody HMB-50 define gene products preferentially expressed in retinal pigment epthelium, Exp Eye Res 1992 November; 55 (5): 657-62). It was isolated from the retinal epithelium. The sequence RPE1 (FIG. 3) published by Kim and Wiston probably corresponds to a partial sequence of the transcript of the bovine SILVER gene or to an isoform of the same gene, expressed at the level of the retina. In fact it lacks (FIG. 3) 499 bp of the 5′ region, precisely the region where the Charolais mutation is situated. The sequence also differs from that which we identified for the SILVER gene by: the substitution of the adenine in position 1151 by a cytosine (A1151C); the deletion of the codon CAG in position 1458 (ACAG1458); the substitution of a guanine in position 1461 by an adenine (G1461A) and the substitution of the cytosine in position 1864 by an adenine (C1864A). The positions are cited with respect to the Charolais bovine transcript (FIG. 3).

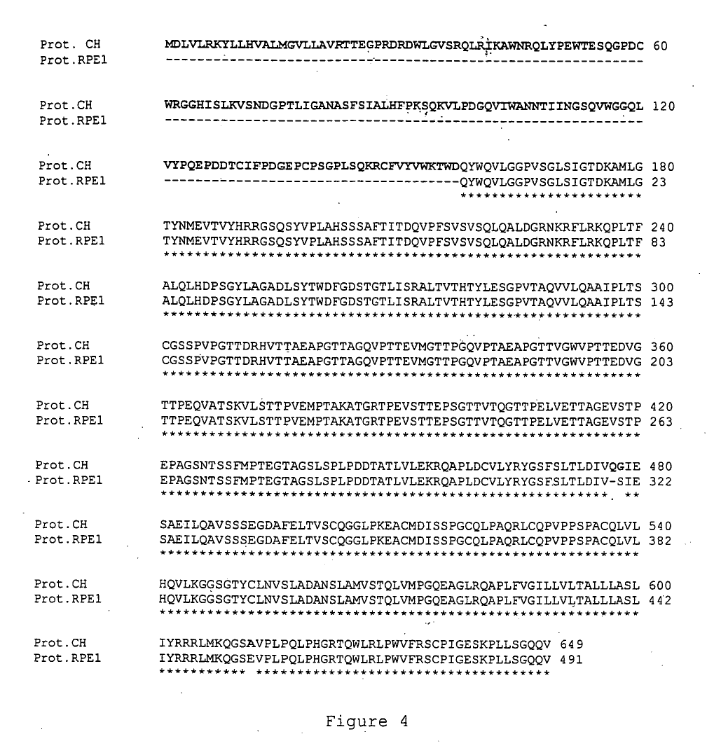

These differences at the level of the nucleotide sequence are expressed by modifications at the peptide level (FIG. 4). The ΔCAG1458 deletion leads to the deletion of the amino acid glutamine in position 477 (ΔQ477); the G1461A substitution, replacing the amino acid glycine with the amino acid serine in position 478 (G478S) and the C1864A substitution, exchanging the amino acid alanine in position 612 for glutamic acid (A612E). The A1151C substitution is silent. The position of the amino acids is cited with respect to the peptide sequence which we determined (FIG. 4).

III) Comments and Discussion

The identified si mutation constitutes the first and only molecular and genetic marker characterizing a single cattle breed, in this instance the Charolais breed to the exclusion of all the other breeds. Its discovery results from analysis of the phenotype and molecular data obtained from mice and humans published by other authors (Berson et al., Mol Biol Cell 2001 12(11): 3451-64; Raposo et al., J. Cell Biol. 2001 Feb. 19; 152 (4):809-24; Kwon et al., Nucleic Acids Res. 1995 January 11.23(1): 154-8; Kwon et al. Proc Natl Acad Sci USA. 1991 Oct. 15; 88 (20): 9228-32; Kim et al. 1992, Exp Eye Res November; 55(5): 657-62.). It takes into account the works relating to the structure of the melanosomes in cattle (Renieri et al., Pigment Cell Res. (1993), 6, 165-170). It is also based on a re-evaluation of the data on murine, human and bovine sequences available in the databases. It is also the outcome of a specific experimental work using the genome of the cattle breeds selected by professionals on the basis of their coat-coloration phenotype.

The genetic marker which is the subject of this innovation characterizes the allele of the bovine SILVER gene (also called PMEL17, GPI00 or ME20M). The silver gene in mice participates in the biogenesis of the melanosome and this is also probable in the other mammals. The mutation of the bovine gene PMEL17 (SILVER) identifies the latter as the dilution gene, thus previously named on the basis of numerous observations resulting from analysis of the phenotypes of cross-bred animals, one of the parents being a pure-bred Charolais (registered in the Herd Book).

The Charolais si allele of the PMEL17 gene is characterized by the substitution of the guanine in position 64 (FIG. 1) by an adenine (G64A). This substitution modifies the codon GGG in position 22, specifying the amino acid glycine or G, in a codon AGG specifying arginine or R (nucleotide No. 1 corresponds to the adenine of the start codon ATG which specifies methionine or M; FIG. 1).

The Charolais mutation G64A affects the peptide signal of the protein PMEL 17, at a site essential for its correct maturation in the endoplasmic reticulum of the melanocyte. Thus, the incorrectly matured PMEL 17 protein can no longer engage in the normal route towards the melanosome via the Golgi apparatus, an organelle in which glycosylation is carried out. The mutated PMEL 17 cannot therefore, in a correct form, finally reach the melanosome where its normal function is to participate in the matrix on which the polymerisation of the melanines and their fixation are carried out. The Charolais melanosome is therefore not, or only slightly coloured, because of the structural lack or absence of the PMEL17 protein. In the homozygous state, the two alleles of the SILVER gene are mutated, which has the consequence that all the PMEL17 proteins are ineffective in the polymerisation and deposition of melanins. Thus the G64A mutation leads to the formation of the “creamy white” colour, typical of Charolais cattle. In the heterozygous state, a single allele is mutated which probably results in only one half of the PMEL17 proteins being ineffective. The absence of part of the matrix of the melanosome is manifested by a dilution of the colour of the cross-bred animal.

In the case of the second si₁ allele, three nucleotides (ΔT53, ΔT54 and ΔC55) are deleted in the region coding the peptide signal. This si₁ allele, leads precisely to the deletion of a hydrophobic amino acid (leucine). Because of the position of this deletion (adjacent to the Charolais mutation) in addition to the role of the amino acid (leucine) in the effectiveness of elimination of the peptide signal, this second allele of the SILVER gene will have a similar effect to the Charolais allele: a dilution of the coat colour. We genotyped 12 individuals of the French Simmental breed. 5 individuals are si₁/si₁ homozygous, 5 others are SI/si₁ heterozygous and two possess the wild-type allele in the homozygous state (SI/SI).

The Simmental breed has the same common ancestor dating from the Middle Ages, the place of origin of which is situated in the Simme river Valley in Switzerland, in the Bernese Oberland (Canton of Berne).

The heterozygoty presented by the French Simmental breed is not surprising to the extent that it forms part of the dairy breeds in France which are very open to the outside, where the proportion of foreign genes reaches or exceeds 75% (Boichard et al. 1996, INRA Prod. Anim., 9 (5), 323-335).

1-19. (canceled)

20. A method for the identification of populations or breeds of ruminant mammals such as cattle, sheep and goats, said method being carried out starting with a biological sample collected from the animal, in particular starting with sperm, embryo, blood, milk, hairs, carcass or meat, or other products derived from the latter, and making it possible to check, or even to certify, whether or not the animal from which said biological sample was collected belongs to a population or breed of ruminant mammals, this method comprising:
a stage of amplification of the number of copies of the different allelic forms of the SILVER gene, namely of the SI, and/or si, and/or si₁ alleles, and/or of fragments of these allelic forms, specific to a population or breed of specific ruminant mammals, and capable of being present in said biological sample,
a stage of detection of said allelic forms or fragments of the latter.

21. The method of claim 21, for the identification of different cattle populations or breeds, or of different cattle herds each including several cattle populations or breeds, comprising a stage of amplification of the number of copies of the different allelic forms of the SILVER gene represented by SEQ ID NO: 1, coding for the bovine SI protein represented by SEQ ID NO: 2, and/or of fragments of this gene represented by SEQ ID NO: 1 or of its different allelic forms.

22. The method according to claim 20, making it possible to check, or even to certify, that an animal belongs to a particular cattle population or breed, or to a particular cattle herd, or, on the other hand, making it possible to certify the exclusion of this animal from this population or breed, or from this particular herd.

23. The method according to claim 20, characterized in that cattle populations or breeds or cattle herds are of French origin.

24. The method according to claim 20, comprising the amplification:
of the nucleotide sequence corresponding to the si allelic form represented by SEQ ID NO: 3, coding for the bovine si protein represented by SEQ ID NO: 4, or corresponding to fragments of this allelic form, said allelic form comprising the G93A mutation with respect to the SI gene, said method making it possible to check, or even to certify, that an animal belongs to the Charolais breed,
of the nucleotide sequence corresponding to the si₁ allelic form, represented by SEQ ID NO: 5, coding for the bovine si₁ protein represented by SEQ ID NO: 6, or corresponding to fragments of this allelic form, said allelic form comprising a deletion of the three nucleotides TTC situated in positions 82, 83 and 84 with respect to the SI gene, said method making it possible to check, or even to certify, that an animal belongs to the Simmental breed,
of the nucleotide sequence corresponding to the bovine SI gene represented by SEQ ID NO: 1, or to fragments of this gene, said method making it possible to certify the exclusion of an animal from the Charolais breed.

25. The method of claim 20, comprising the amplification of fragments of the nucleotide sequences corresponding to the allelic forms, SI, si, and si₁, represented by SEQ ID NO: 1, 3, and 5 respectively, said fragments being chosen from those of approximately 10 to 300 nucleotides containing the nucleotides situated in positions 82 to 93 of said sequences.

26. The method of claim 20, comprising the amplification of fragments of the nucleotide sequences corresponding to the allelic forms SI, si, and si₁, said fragments being chosen from those of 294 nucleotides delimited by the nucleotides situated in positions 9 and 302 of the sequences SEQ ID NO: 1 and 3, these fragments being represented by the sequences SEQ ID NO: 7, and SEQ ID NO: 8 respectively, and the fragment of 291 nucleotides delimited by the nucleotides situated in positions 9 and 299 of the sequence SEQ ID NO: 5, this fragment being represented by the sequence SEQ ID NO: 9.

27. The method of claim 20, comprising the amplification by nucleotide primers of the number of copies of the SILVER gene, or of the different allelic forms of this gene, or of the fragments of this gene or of its different allelic forms.

28. The method of claim 20, comprising the amplification by nucleotide primers of the number of copies of the SILVER gene, or of the different allelic forms of this gene, or of the fragments of this gene or of its different allelic forms, wherein said nucleotide primers are in the form of 5′-3′ primer pairs, these pairs being such that:
the 5′ primer is chosen from the SIL10 primer represented by the following sequence SEQ ID NO: 10:
5′ GTTGCTGGAAGGAAGAACAGGATGGATCTG 3′
or any sequence derived from this sequence SEQ ID NO: 10, in particular by suppression and/or substitution and/or addition of one or more nucleotides, said derived sequence being hybridized, like the sequence SEQ ID NO: 10, with all or part of the nucleotide sequence complementary to the nucleotide sequences delimited by the nucleotides situated in positions 9 and 38 of the sequences SEQ ID NO: 1, 3, and 5,
the 3′ primer is chosen from the SIL8 primer represented by the following sequence SEQ ID NO: 11:
5′ CAGTCCCAAGTGCCTGAACACACATGCACC 3′
or any sequence derived from this sequence SEQ ID NO: 11, in particular by suppression and/or substitution and/or addition of one or more nucleotides, said derived sequence being hybridized, like the sequence SEQ ID NO: 11, with all or part of the nucleotide sequences delimited by the nucleotides situated in positions 276 and 302 of the sequences SEQ ID NO: 1, 3, and by the nucleotides situated in positions 273 and 299, of the sequence SEQ ID NO: 5.

29. A nucleotide sequence characterized in that it corresponds to the bovine SI gene represented by SEQ ID NO:1, or to the following fragments of the SI gene:
any fragment chosen from those of approximately 10 to 300 nucleotides containing the nucleotides situated in positions 82 to 93 of the sequence SEQ ID NO: 1,
the fragment SEQ ID NO: 7 of 294 nucleotides delimited by the nucleotides situated in positions 9 and 302 of the sequence SEQ ID NO: 1.

30. A nucleotide sequence characterized in that it corresponds to the bovine si gene represented by SEQ ID NO: 3, or to the following fragments of the si gene:
any fragment chosen from those of approximately 10 to 300 nucleotides containing the nucleotides situated in positions 82 to 93 of the sequence SEQ ID NO: 3,
the fragment SEQ ID NO: 8 of 294 nucleotides delimited by the nucleotides situated in positions 9 and 302 of the sequence SEQ ID NO: 3.

31. A nucleotide sequence characterized in that it corresponds to the bovine si₁ gene represented by SEQ ID NO: 5, or to the following fragments of the si₁ gene:
any fragment chosen from those of approximately 10 to 300 nucleotides containing the nucleotides situated in positions 82 to 93 of the sequence SEQ ID NO: 5,
the fragment SEQ ID NO: 9 of 291 nucleotides delimited by the nucleotides situated in positions 9 and 299 of the sequence SEQ ID NO: 5.

32. A nucleotide sequence characterized in that it comprises:
the SIL10 nucleotide sequence represented by the following sequence
SEQ ID NO: 10:
5′ GTTGCTGGAAGGAAGAACAGGATGGATCTG 3′
or any sequence derived from this sequence SEQ ID NO: 10, in particular by suppression and/or substitution and/or addition of one or more nucleotides, said derived sequence being hybridized, like the sequence SEQ ID NO: 10, with all or part of the nucleotide sequence complementary to the nucleotide sequences delimited by the nucleotides situated in positions 9 and 38 of the sequences SEQ ID NO: 1, 3, and 5,
the SIL8 nucleotide sequence represented by the following sequence
SEQ ID NO: 11:
5′ CAGTCCCAAGTGCCTGAACACACATGCACC 3′
or any sequence derived from this sequence SEQ ID NO: 11, in particular by suppression and/or substitution and/or addition of one or more nucleotides, said derived sequence being hybridized, like the sequence SEQ ID NO: 11, with all or part of the nucleotide sequences delimited by the nucleotides situated in positions 276 and 302 of the sequences SEQ ID NO: 1, 3, and by the nucleotides situated in positions 273 and 299, of the sequence SEQ ID NO: 5.

33. Primer pairs, each of the two primers comprising, independently of one other, approximately 10 to approximately 30 nucleotides, characterized in that they are chosen in such a manner that one of the two sequences of a primer pair is hybridized with a sequence of approximately 10 to approximately 30 nucleotides comprised in the nucleotide sequence complementary to the sequence delimited by the nucleotides situated in positions 1 and approximately 60 of the nucleotide sequences SEQ ID NO: 1, 3, and 5, whilst the other sequence of this same pair is hybridized with a sequence of approximately 10 to approximately 30 nucleotides comprised between the nucleotide situated in position 94 and the last of the nucleotides of the sequences SEQ ID NO: 1, 3, and 5.

34. Primer pairs for gene amplification according to claim 33, characterized in that:
one of the primers is chosen from the sequences comprising the SIL10 sequence represented by SEQ ID NO: 10, or any sequence derived from this sequence SEQ ID NO: 10, in particular by suppression and/or substitution and/or addition of one or more nucleotides, said derived sequence being hybridized, like the sequence SEQ ID NO: 10, with all or part of the nucleotide sequence complementary to the nucleotide sequences delimited by the nucleotides situated in positions 9 and 38 of the sequences SEQ ID NO: 1, 3, and 5,
said primer being advantageously labelled, in particular in a radioactive or fluorescent manner,
whilst the other primer is chosen from the sequences comprising the SIL8 sequence represented by SEQ ID NO: 11, or any sequence derived from this sequence SEQ ID NO: 11, in particular by suppression and/or substitution and/or addition of one or more nucleotides, said derived sequence being hybridized, like the sequence SEQ ID NO: 11, with all or part of the nucleotide sequences delimited by the nucleotides situated in positions 276 and 302 of the sequences SEQ ID NO: 1, 3, and by the nucleotides situated in positions 273 and 299, of the sequence SEQ ID NO: 5.

35. A method for the identification of populations or breeds of ruminant mammals such as cattle, sheep and goats, said method being carried out starting with a biological sample collected from the animal, in particular starting with sperm, embryo, blood, milk, hairs, carcass or meat, or other products derived from the latter, and making it possible to check, or even to certify, whether or not the animal from which said biological sample was collected belongs to a population or breed of ruminant mammals, this method comprising:
a stage of amplification of the number of copies of the different allelic forms of the SILVER gene, namely of the SI, and/or si, and/or si₁ alleles, and/or of fragments of these allelic forms, specific to a population or breed of specific ruminant mammals, and capable of being present in said biological sample,
a stage of detection of said allelic forms or fragments of the latter,
characterized in that the stage of amplification of the number of copies of the different allelic forms of the SILVER gene, or of the fragments of these allelic forms, is carried out using a primer pair as defined in claim 33.

36. A method for the identification of populations or breeds of ruminant mammals such as cattle, sheep and goats, said method being carried out starting with a biological sample collected from the animal, in particular starting with sperm, embryo, blood, milk, hairs, carcass or meat, or other products derived from the latter, and making it possible to check, or even to certify, whether or not the animal from which said biological sample was collected belongs to a population or breed of ruminant mammals, this method comprising:
a stage of amplification of the number of copies of the different allelic forms of the SILVER gene, namely of the SI, and/or si, and/or si₁ alleles, and/or of fragments of these allelic forms, specific to a population or breed of specific ruminant mammals, and capable of being present in said biological sample,
a stage of detection of said allelic forms or fragments of the latter,
characterized in that the stage of amplification of the number of copies of the different allelic forms of the SILVER gene, or of the fragments of these allelic forms, is carried out using a primer pair as defined in claim 33,
said method being characterized in that:
the detection of a genotype comprising the si allele in the biological sample studied makes it possible to certify that said sample originates from an animal belonging to the Charolais breed or having at least one ancestor of the Charolais breed,
the detection of a genotype comprising the si₁ allele in the biological sample studied makes it possible to certify that said sample originates from an animal belonging to the Simmental breed or having at least one ancestor of the Simmental breed,
the detection of a genotype comprising the SI allele, makes it possible to certify that said sample does not originate from an animal of the Charolais breed.

37. A kit for the implementation of a method according to claim 20, characterized in that it comprises at least one primer pair wherein each of the two primers comprising, independently of one other, approximately 10 to approximately 30 nucleotides, characterized in that they are chosen in such a manner that one of the two sequences of a primer pair is hybridized with a sequence of approximately 10 to approximately 30 nucleotides comprised in the nucleotide sequence complementary to the sequence delimited by the nucleotides situated in positions 1 and approximately 60 of the nucleotide sequences SEQ ID NO: 1, 3, and 5, whilst the other sequence of this same pair is hybridized with a sequence of approximately 10 to approximately 30 nucleotides comprised between the nucleotide situated in position 94 and the last of the nucleotides of the sequences SEQ ID NO: 1, 3, and 5, and if appropriate the reagents necessary for the implementation of the amplification reaction of the number of copies of the different allelic forms of the SILVER gene.


SEQUENCE LISTING (11 sequences)

SEQ ID NO: 1 - DNA, 8146 bp, Bos taurus
  CDS (30)..(105)
ggtctttggttgctggaaggaagaacaggatggatctggtgctgagaaaataccttctccatgtggctctgatgggtgttcttctggctgtagggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactgaacctgaaaacaaccccatctacctggatgggtgagagaacagtatgtctccgtggccctaattttgagatgctctgaatagtgagctggaacatgggtgccaaggtagtaaaatgagtggaaactcatttaggctttgtctcaggcacttgggatagggtatttaggagatagagaaagataggagataggagaaaggagaaagaggatgtggtattggatagaagggtaatgaggcacctcatcccctctttgggatgggcatgggtgaacacagcccaggcttttgttctggggctggaagagacaggcagaagggtctcagctgagcatcacatgaaagggctctgggggattggggcctcgtgacaggagcaaggcgggtggggtggggatggtgagagggtctggaatgtcccgtgctgctctgaggagggaggattgggagtggagaaagaatggggcatcttatgattctcttgttcttgtggtgaggtattcagtgggataattctagatcctcccccaagagaatcaaccaggtttctggtacatgttagagatggagtgaggatagtctgtgatgtgcagaaatatctacattgtaccccagtgccccctttctctagatccctggtctcacagacttcttggaacttctccttgatctgacttccctcattcatggtgtcatttcaagtcttattcttttactatgttcgttattgtattctggaaatatcctgttcatatgtgtccacccaaggctcttaatatgttgtgcttactttttggatccagatttttaaaatcataagaagacatttttatatagttcatgaaattttgcatggactgagtttgataattttgtttagtgtgaattaacattgtgtttatttaagaaaaaaaaatatttttttacagaaacctactgaatttgtagggttttaaaataacatgatgtctgggatttgcttttgaatgcttcagccaaaaaacaaacgaacaacaaaaataaaggatagataaagcaaatgtgacaaaatgctgatagttgttggaccttggggagacacatgcagagccatcacatcactttttttcagacatctttcttggtcagttataatcattttgtttgtccccactcccaatttctacttgcctctagtccatcctcctcactgcttgccaaagtgatccttctaaaacacaaatctgatcatattcaaaaagcttttgaagggtaagttttatggtatatgccatatatcagtacaacaaacaaaatcgtctgaggtgccgttgcctacaggataaagtccaaactcctttgcctggcactccaagcccccactctatcttcttggcctcatctctcatgatgtacatcagccacattgctagtgtctgctcatggccttctgcctagaatgctttatgccccagccaactatttactgtcttcttcagtcgaccagagtgcaatttacctgtttaaaatctatcattttgttatacattgtgcatgtctattatggctcatattaagcaatgccttggattatagtaatttatgtatatgtctatttcatatactttaacctgaaccccttcagaaccatttctttttcatttcttaagttctttgcacctagcccagtgcctggtacgtcgtgggtattcagtagattaaaatgcactttaaggaacttcccttgttgtccatcaagtggctaaggctctgtgctcccaatgcaggggaccagggttcaatctcaggtcagggaactagatcccacaggtcacaactaagagtttgcaagccacaactacctgacctcacatgccacaactaatcgaagatccctcgtgctgcaactaagtcctagtgcagttaaatatatttttttaatgcactttgaatgtgagaatgaatgatgtgtcacagacactgttgtcccctgagaagggagtgagtaatgatttgagggccctcatagtatatcttcctttttaggacccagagacagggactggcttggtgtctcaaggcagctcagaattaaagcatggaacagacagctgtatccagagtggacagaaagccaggggcctgactgctggagaggtaggaacttggcaatttccagggaggatatggtggaaatgggtggggaggggaacggggttgaatgtacttaggaagatagggaaggaaaaggcatacagggaggagaagccaaggagctaattaatgcagctgcccttttcaggtggccacatatccctgaaggtcagcaatgatgggcctacactgattggggcaaatgcttccttctctattgccttgcactttcctaaaagccaaaaggtgctgccagatgggcaggtcatctgggccaacaacaccatcatcaatggtgagtacctctccgcctccttcccaaggtccagaatccctggtatccccaatgagctcaaggaatcctcctcctctttttttttttttttttttacaaattatatatgtaacacatattcactgcagaaaaattagaaaacacagataaaccaaaaagaaaaaaaattatagttccccaaatggggcacagaagacccagtggacatagaagttggatagacttggatttaaactggttaccagtatgtgaccctggacaagtcactgaattgttttgttcttccattcccttatctatagaatggggatgataacactttaaaaggttcttgtaaggattaaaatgtgataatatataaagattttagcataatgcctgccctgtgctgtgcttagtaccttagtttagacgctttgcaaccccatggactgtagcccaccaggctcctctgtccatgtggattctgcaggcaagaatactggagtgggtcaccatgcactcctccaggggatcttcccaactcagggatcgaacccaggtcctagcctacagtattaattgatgctgttatttttacttttatcccactagctagagcacatcatcctagacattttgatacatggcctaccaatttgtgtccagtgtaagaatatacatgtgtgtgctcagtggctcagtcgtgtctgactctttgcaaccccatggactgtagcccgcgaaagctcctctgcccatgggattgcccagccaagaatactggagcaggttgccatttcttcctccaggggatctttcaacacagggattgaatccttgtctcctgtgtttcctgcattggcaggtgtattctttaccactgagccacctgggaaaccccttaagtatatacacataaatcttttatagtttccattctcccttctaccactccaaataggttataccaaggagaatgtattttggtagctaggcagtattcctggagcccctctctgggagtcatgttaaaggttttggtgtacagtgaggaatgccagggattgagggagacttgctgtcttcttttcagggagccaggtgtggggaggacagctggtatatccccaagaacctgatgatacctgcatcttccccgatggggagccctgcccttctggccctctatctcagaaaagatgctttgtttatgtctggaagacctggggtaagagtttcccttctctggcctgtcattcacacttaaattcacttcttcctacctgatcccctttcttttggtctcatccttaaattctgtgagtttccctaatcttcacttcccccatgactccttcctcttccacagcacctagtcaactctattatacttctttctgggagccctgctccaattatagtcccatcccatggaccctctcataaggacttttttcctgcccaacatatgcaagcttaaactctctgaaataaccatccttgatacatctcctgaccttccttctctggttccatctctaaccctgccccagtctcctttgaccagtaacccccttccctactcttctttccaaaaacctcagaccaatactggcaagttctggggggcccagtgtctggactgagcatcgggacagacaaggcaatgctgggcacatataacatggaagtgactgtctaccaccgccgggggtcccagagctatgtgcccctcgctcactccagttcagccttcaccattactggtaaggactgaggaggggacaaggccagttgcagggcaggagaaggtggggaggctgggctggacaggaaaggggaaagaggaaatggtgtgtaaccttacaggggcagaaccaggaagatgtgggcagagggatgtggggcttggagcccgtgaagggccaggcagcttgggttggttgaaaaatatggctgtgaaagaagaagctgacagaaagaagaacttatggttctcactttctctgactccaatcccagaccaggtgcccttctctgtgagtgtgtctcagctgcaggccttggatggaaggaacaagcgcttcctgagaaagcagcctctgacctttgccctccagctccatgatcccagtggctatttggctggggctgacctttcctacacctgggactttggtgacagtacagggaccctgatctctcgggcactcacggtcactcacacttacctagagtctggcccagtcactgcacaggtggtgctgcaggctgccattcctctcacctcctgtggctcctctccagttccaggcactacagataggcatgtgacaactgcagaggctcctggaaccacagctggccaagtgcctactacagaagtcatgggcaccacacctggccaggtgccaactgcagaggcccctggcaccacagttgggtgggtgccaaccacagaggatgtaggtaccacacctgagcaggtggcaacctccaaagtcttaagtacaacaccagtggagatgccaactgcaaaagctacaggtaggacacctgaagtgtcaactacagagccctctggaaccacagttacacagggaacaactccagagctggtggagaccacagctggagaggtgtccactcctgagcctgcgggttcaaatactagctcattcatgcctacagaaggtactgcaggtaagggggccaccatgaatgagttcatagaggtggggcatttgtcacagctctgaagacctgaaagaattgctcaggacccagatgttactcaatccttagcttagcagtggagtcccctcagaatcttcactggttttaaaaccccctaagtccctcttaatggcacagaatagatccagagttcaggaaaccagggtcttctcctaggccaggggtagagagcttattctctcttcctgaagagaagttcaggaagcagtgtgtgatcatttggtggtggtgctcagtcatgtctgactctttgtgacctcatggactatggcccaccaggctcctctgtccatagaattctccaggcaagaacactggagtgggtggccatttccttctccaggggattttccctgcccagggattaaacccgaattggcaggtggattctttacccgagccacctagaaagtcccatgtgatcattagataatacttatacctcattttctgattaagtgtaaacacagaaatctttctgacaccacttcccacccctggattcccatcccaaagtaggtttacctggaattgtggtaggaatactaaaaagggagaagtgagatagtgacactatgacttaacacatgtcaaatgtctgacccaggacctggcacagtgtagggtgtgataaacatttgggatgtctaaaattctgactctaaccctgtgactctggggcagtcatttctcttgggcctttctttatcttaaaaaatgagagtttccagctcttgtctgattctaagcctggatccagtagctctgactctacctggaaaaatgcttgttgggcctgttttcaggttagtcatttgctttttgactttgcctctttaatcctctcctccaggctccctgagtcccctgccggatgacactgccaccttagtcctggagaagcgccaagcccccctggattgtgttctgtatcgctatggctccttttccctcaccctggacattgtccgtgagtcttgcctacattgtccgtaagctggtggagggaggcgtgtgctgcttagggttgcccagtggaagcacaccttggaaggaattactcacctggacaaggagaatacccagatcccaggggtttcatatgaaggcagaatgggattagggaggcagcccgaggaccttcctggccatgggccttgggggaggataagtagaggagtctcagacttaaaaaaatcttgcaactttgcagagggtattgagagtgctgagatcctacaggctgtgtcatccagtgaaggagatgcatttgagctgactgtgtcttgccaaggcgggtgagtgtcccacggttgccctgagaactcctggggtgactgctgtcctgttctctggtgtctagtgtcccttcccagattccctgacgtaagctgacatctctcccaggctacccaaggaagcctgcatggacatctcatcgccagggtgtcagctgcctgcccagcggctgtgtcagcctgtgccccccagcccagcctgccagctggttttgcaccaggtactgaagggtggctcagggacctactgcctcaatgtgtctttggctgatgccaatagcctggcgatggtcagcacccagcttgtcatgcctggtaggtagttggacaagaggtaggatgaagacacggggagatggtagaggttacctactagaggaagcagacactgaatgcagccgtatctgggattccacccatagggcaagaagcaggcctcaggcaggctcctctgttcgtgggcatcttgctggtgctaacagctttgttgcttgcatctctgatatacaggtgagatccccgccatcctgctcccactcctttaccccttattaccaccaccactcttcctcatgggaagaagaaaccaccaacccctttgggaaagtgtagagtccaagaaagagcccagacttggaagttcaacaggtctaggctgcagtcttgctggtgggaccctggggaagtccattaacccttctgagccactgaaaagtaggaaacataatacctgtcctgtggggctgttttcagggctctagacaatgtgagtaaaacacctggttctgaaacaaaagtggaataaatgatgatctcaatgactgttgttatgaataatatcaacagtggagaagaactcagtgaactgagttctccacctgccagaaaggcaaatccctaggcctggagggctgaggtcctcaaagcagggaagcctgtagggtgagagggaaatggtcagagcttaccataaacataagagaggataaaccctgttggtgagaagaggagggagccaggatcaagaccaagtcaacctgggttatggtttagtcttttttttttagagaagcacaaagaggttgccattgaccaccactaaccagtatccctgcttttctcccaatatcaggcgaagacttatgaagcaaggctcagcagtcccccttccccagctgccacacggtagaacccagtggctacgtctgccctgggtcttccgctcttgccccattggtgagagcaaacccctcctcagtggacagcaggtctgagtgctcttatgtgaagtcatgatttacccaggtggacagcaaggcctgtcttttctctggtcttccctcagagactaccattgcctgaaataaagactcagaacttg

SEQ ID NO: 2 - protein, 649 aa, Bos taurus
MDLVLRKYLLHVALMGVLLAVGTTEGPRDRDWLGVSRQLRIKAWNRQLYPEWTESQGPDCWRGGHISLKVSNDGPTLIGANASFSIALHFPKSQKVLPDGQVIWANNTIINGSQVWGGQLVYPQEPDDTCIFPDGEPCPSGPLSQKRCFVYVWKTWDQYWQVLGGPVSGLSIGTDKAMLGTYNMEVTVYHRRGSQSYVPLAHSSSAFTITDQVPFSVSVSQLQALDGRNKRFLRKQPLTFALQLHDPSGYLAGADLSYTWDFGDSTGTLISRALTVTHTYLESGPVTAQVVLQAAIPLTSCGSSPVPGTTDRHVTTAEAPGTTAGQVPTTEVMGTTPGQVPTAEAPGTTVGWVPTTEDVGTTPEQVATSKVLSTTPVEMPTAKATGRTPEVSTTEPSGTTVTQGTTPELVETTAGEVSTPEPAGSNTSSFMPTEGTAGSLSPLPDDTATLVLEKRQAPLDCVLYRYGSFSLTLDIVQGIESAEILQAVSSSEGDAFELTVSCQGGLPKEACMDISSPGCQLPAQRLCQPVPPSPACQLVLHQVLKGGSGTYCLNVSLADANSLAMVSTQLVMPGQEAGLRQAPLFVGILLVLTALLLASLIYRRRLMKQGSAVPLPQLPHGRTQWLRLPWVFRSCPIGESKPLLSGQQV

SEQ ID NO: 3 - DNA, 8146 bp, Bos taurus
  CDS (30)..(105)
ggtctttggttgctggaaggaagaacaggatggatctggtgctgagaaaataccttctccatgtggctctgatgggtgttcttctggctgtaaggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactgaacctgaaaacaaccccatctacctggatgggtgagagaacagtatgtctccgtggccctaattttgagatgctctgaatagtgagctggaacatgggtgccaaggtagtaaaatgagtggaaactcatttaggctttgtctcaggcacttgggatagggtatttaggagatagagaaagataggagataggagaaaggagaaagaggatgtggtattggatagaagggtaatgaggcacctcatcccctctttgggatgggcatgggtgaacacagcccaggcttttgttctggggctggaagagacaggcagaagggtctcagctgagcatcacatgaaagggctctgggggattggggcctcgtgacaggagcaaggcgggtggggtggggatggtgagagggtctggaatgtcccgtgctgctctgaggagggaggattgggagtggagaaagaatggggcatcttatgattctcttgttcttgtggtgaggtattcagtgggataattctagatcctcccccaagagaatcaaccaggtttctggtacatgttagagatggagtgaggatagtctgtgatgtgcagaaatatctacattgtaccccagtgccccctttctctagatccctggtctcacagacttcttggaacttctccttgatctgacttccctcattcatggtgtcatttcaagtcttattcttttactatgttcgttattgtattctggaaatatcctgttcatatgtgtccacccaaggctcttaatatgttgtgcttactttttggatccagatttttaaaatcataagaagacatttttatatagttcatgaaattttgcatggactgagtttgataattttgtttagtgtgaattaacattgtgtttatttaagaaaaaaaaatatttttttacagaaacctactgaatttgtagggttttaaaataacatgatgtctgggatttgcttttgaatgcttcagccaaaaaacaaacgaacaacaaaaataaaggatagataaagcaaatgtgacaaaatgctgatagttgttggaccttggggagacacatgcagagccatcacatcactttttttcagacatctttcttggtcagttataatcattttgtttgtccccactcccaatttctacttgcctctagtccatcctcctcactgcttgccaaagtgatccttctaaaacacaaatctgatcatattcaaaaagcttttgaagggtaagttttatggtatatgccatatatcagtacaacaaacaaaatcgtctgaggtgccgttgcctacaggataaagtccaaactcctttgcctggcactccaagcccccactctatcttcttggcctcatctctcatgatgtacatcagccacattgctagtgtctgctcatggccttctgcctagaatgctttatgccccagccaactatttactgtcttcttcagtcgaccagagtgcaatttacctgtttaaaatctatcattttgttatacattgtgcatgtctattatggctcatattaagcaatgccttggattatagtaatttatgtatatgtctatttcatatactttaacctgaaccccttcagaaccatttctttttcatttcttaagttctttgcacctagcccagtgcctggtacgtcgtgggtattcagtagattaaaatgcactttaaggaacttcccttgttgtccatcaagtggctaaggctctgtgctcccaatgcaggggaccagggttcaatctcaggtcagggaactagatcccacaggtcacaactaagagtttgcaagccacaactacctgacctcacatgccacaactaatcgaagatccctcgtgctgcaactaagtcctagtgcagttaaatatatttttttaatgcactttgaatgtgagaatgaatgatgtgtcacagacactgttgtcccctgagaagggagtgagtaatgatttgagggccctcatagtatatcttcctttttaggacccagagacagggactggcttggtgtctcaaggcagctcagaattaaagcatggaacagacagctgtatccagagtggacagaaagccaggggcctgactgctggagaggtaggaacttggcaatttccagggaggatatggtggaaatgggtggggaggggaacggggttgaatgtacttaggaagatagggaaggaaaaggcatacagggaggagaagccaaggagctaattaatgcagctgcccttttcaggtggccacatatccctgaaggtcagcaatgatgggcctacactgattggggcaaatgcttccttctctattgccttgcactttcctaaaagccaaaaggtgctgccagatgggcaggtcatctgggccaacaacaccatcatcaatggtgagtacctctccgcctccttcccaaggtccagaatccctggtatccccaatgagctcaaggaatcctcctcctctttttttttttttttttttacaaattatatatgtaacacatattcactgcagaaaaattagaaaacacagataaaccaaaaagaaaaaaaattatagttccccaaatggggcacagaagacccagtggacatagaagttggatagacttggatttaaactggttaccagtatgtgaccctggacaagtcactgaattgttttgttcttccattcccttatctatagaatggggatgataacactttaaaaggttcttgtaaggattaaaatgtgataatatataaagattttagcataatgcctgccctgtgctgtgcttagtaccttagtttagacgctttgcaaccccatggactgtagcccaccaggctcctctgtccatgtggattctgcaggcaagaatactggagtgggtcaccatgcactcctccaggggatcttcccaactcagggatcgaacccaggtcctagcctacagtattaattgatgctgttatttttacttttatcccactagctagagcacatcatcctagacattttgatacatggcctaccaatttgtgtccagtgtaagaatatacatgtgtgtgctcagtggctcagtcgtgtctgactctttgcaaccccatggactgtagcccgcgaaagctcctctgcccatgggattgcccagccaagaatactggagcaggttgccatttcttcctccaggggatctttcaacacagggattgaatccttgtctcctgtgtttcctgcattggcaggtgtattctttaccactgagccacctgggaaaccccttaagtatatacacataaatcttttatagtttccattctcccttctaccactccaaataggttataccaaggagaatgtattttggtagctaggcagtattcctggagcccctctctgggagtcatgttaaaggttttggtgtacagtgaggaatgccagggattgagggagacttgctgtcttcttttcagggagccaggtgtggggaggacagctggtatatccccaagaacctgatgatacctgcatcttccccgatggggagccctgcccttctggccctctatctcagaaaagatgctttgtttatgtctggaagacctggggtaagagtttcccttctctggcctgtcattcacacttaaattcacttcttcctacctgatcccctttcttttggtctcatccttaaattctgtgagtttccctaatcttcacttcccccatgactccttcctcttccacagcacctagtcaactctattatacttctttctgggagccctgctccaattatagtcccatcccatggaccctctcataaggacttttttcctgcccaacatatgcaagcttaaactctctgaaataaccatccttgatacatctcctgaccttccttctctggttccatctctaaccctgccccagtctcctttgaccagtaacccccttccctactcttctttccaaaaacctcagaccaatactggcaagttctggggggcccagtgtctggactgagcatcgggacagacaaggcaatgctgggcacatataacatggaagtgactgtctaccaccgccgggggtcccagagctatgtgcccctcgctcactccagttcagccttcaccattactggtaaggactgaggaggggacaaggccagttgcagggcaggagaaggtggggaggctgggctggacaggaaaggggaaagaggaaatggtgtgtaaccttacaggggcagaaccaggaagatgtgggcagagggatgtggggcttggagcccgtgaagggccaggcagcttgggttggttgaaaaatatggctgtgaaagaagaagctgacagaaagaagaacttatggttctcactttctctgactccaatcccagaccaggtgcccttctctgtgagtgtgtctcagctgcaggccttggatggaaggaacaagcgcttcctgagaaagcagcctctgacctttgccctccagctccatgatcccagtggctatttggctggggctgacctttcctacacctgggactttggtgacagtacagggaccctgatctctcgggcactcacggtcactcacacttacctagagtctggcccagtcactgcacaggtggtgctgcaggctgccattcctctcacctcctgtggctcctctccagttccaggcactacagataggcatgtgacaactgcagaggctcctggaaccacagctggccaagtgcctactacagaagtcatgggcaccacacctggccaggtgccaactgcagaggcccctggcaccacagttgggtgggtgccaaccacagaggatgtaggtaccacacctgagcaggtggcaacctccaaagtcttaagtacaacaccagtggagatgccaactgcaaaagctacaggtaggacacctgaagtgtcaactacagagccctctggaaccacagttacacagggaacaactccagagctggtggagaccacagctggagaggtgtccactcctgagcctgcgggttcaaatactagctcattcatgcctacagaaggtactgcaggtaagggggccaccatgaatgagttcatagaggtggggcatttgtcacagctctgaagacctgaaagaattgctcaggacccagatgttactcaatccttagcttagcagtggagtcccctcagaatcttcactggttttaaaaccccctaagtccctcttaatggcacagaatagatccagagttcaggaaaccagggtcttctcctaggccaggggtagagagcttattctctcttcctgaagagaagttcaggaagcagtgtgtgatcatttggtggtggtgctcagtcatgtctgactctttgtgacctcatggactatggcccaccaggctcctctgtccatagaattctccaggcaagaacactggagtgggtggccatttccttctccaggggattttccctgcccagggattaaacccgaattggcaggtggattctttacccgagccacctagaaagtcccatgtgatcattagataatacttatacctcattttctgattaagtgtaaacacagaaatctttctgacaccacttcccacccctggattcccatcccaaagtaggtttacctggaattgtggtaggaatactaaaaagggagaagtgagatagtgacactatgacttaacacatgtcaaatgtctgacccaggacctggcacagtgtagggtgtgataaacatttgggatgtctaaaattctgactctaaccctgtgactctggggcagtcatttctcttgggcctttctttatcttaaaaaatgagagtttccagctcttgtctgattctaagcctggatccagtagctctgactctacctggaaaaatgcttgttgggcctgttttcaggttagtcatttgctttttgactttgcctctttaatcctctcctccaggctccctgagtcccctgccggatgacactgccaccttagtcctggagaagcgccaagcccccctggattgtgttctgtatcgctatggctccttttccctcaccctggacattgtccgtgagtcttgcctacattgtccgtaagctggtggagggaggcgtgtgctgcttagggttgcccagtggaagcacaccttggaaggaattactcacctggacaaggagaatacccagatcccaggggtttcatatgaaggcagaatgggattagggaggcagcccgaggaccttcctggccatgggccttgggggaggataagtagaggagtctcagacttaaaaaaatcttgcaactttgcagagggtattgagagtgctgagatcctacaggctgtgtcatccagtgaaggagatgcatttgagctgactgtgtcttgccaaggcgggtgagtgtcccacggttgccctgagaactcctggggtgactgctgtcctgttctctggtgtctagtgtcccttcccagattccctgacgtaagctgacatctctcccaggctacccaaggaagcctgcatggacatctcatcgccagggtgtcagctgcctgcccagcggctgtgtcagcctgtgccccccagcccagcctgccagctggttttgcaccaggtactgaagggtggctcagggacctactgcctcaatgtgtctttggctgatgccaatagcctggcgatggtcagcacccagcttgtcatgcctggtaggtagttggacaagaggtaggatgaagacacggggagatggtagaggttacctactagaggaagcagacactgaatgcagccgtatctgggattccacccatagggcaagaagcaggcctcaggcaggctcctctgttcgtgggcatcttgctggtgctaacagctttgttgcttgcatctctgatatacaggtgagatccccgccatcctgctcccactcctttaccccttattaccaccaccactcttcctcatgggaagaagaaaccaccaacccctttgggaaagtgtagagtccaagaaagagcccagacttggaagttcaacaggtctaggctgcagtcttgctggtgggaccctggggaagtccattaacccttctgagccactgaaaagtaggaaacataatacctgtcctgtggggctgttttcagggctctagacaatgtgagtaaaacacctggttctgaaacaaaagtggaataaatgatgatctcaatgactgttgttatgaataatatcaacagtggagaagaactcagtgaactgagttctccacctgccagaaaggcaaatccctaggcctggagggctgaggtcctcaaagcagggaagcctgtagggtgagagggaaatggtcagagcttaccataaacataagagaggataaaccctgttggtgagaagaggagggagccaggatcaagaccaagtcaacctgggttatggtttagtcttttttttttagagaagcacaaagaggttgccattgaccaccactaaccagtatccctgcttttctcccaatatcaggcgaagacttatgaagcaaggctcagcagtcccccttccccagctgccacacggtagaacccagtggctacgtctgccctgggtcttccgctcttgccccattggtgagagcaaacccctcctcagtggacagcaggtctgagtgctcttatgtgaagtcatgatttacccaggtggacagcaaggcctgtcttttctctggtcttccctcagagactaccattgcctgaaataaagactcagaacttg

SEQ ID NO: 4 - protein, 649 aa, Bos taurus
MDLVLRKYLLHVALMGVLLAVRTTEGPRDRDWLGVSRQLRIKAWNRQLYPEWTESQGPDCWRGGHISLKVSNDGPTLIGANASFSIALHFPKSQKVLPDGQVIWANNTIINGSQVWGGQLVYPQEPDDTCIFPDGEPCPSGPLSQKRCFVYVWKTWDQYWQVLGGPVSGLSIGTDKAMLGTYNMEVTVYHRRGSQSYVPLAHSSSAFTITDQVPFSVSVSQLQALDGRNKRFLRKQPLTFALQLHDPSGYLAGADLSYTWDFGDSTGTLISRALTVTHTYLESGPVTAQVVLQAAIPLTSCGSSPVPGTTDRHVTTAEAPGTTAGQVPTTEVMGTTPGQVPTAEAPGTTVGWVPTTEDVGTTPEQVATSKVLSTTPVEMPTAKATGRTPEVSTTEPSGTTVTQGTTPELVETTAGEVSTPEPAGSNTSSFMPTEGTAGSLSPLPDDTATLVLEKRQAPLDCVLYRYGSFSLTLDIVQGIESAEILQAVSSSEGDAFELTVSCQGGLPKEACMDISSPGCQLPAQRLCQPVPPSPACQLVLHQVLKGGSGTYCLNVSLADANSLAMVSTQLVMPGQEAGLRQAPLFVGILLVLTALLLASLIYRRRLMKQGSAVPLPQLPHGRTQWLRLPWVFRSCPIGESKPLLSGQQV

SEQ ID NO: 5 - DNA, 8143 bp, Bos taurus
  CDS (30)..(102)
ggtctttggttgctggaaggaagaacaggatggatctggtgctgagaaaataccttctccatgtggctctgatgggtgttctggctgtagggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactgaacctgaaaacaaccccatctacctggatgggtgagagaacagtatgtctccgtggccctaattttgagatgctctgaatagtgagctggaacatgggtgccaaggtagtaaaatgagtggaaactcatttaggctttgtctcaggcacttgggatagggtatttaggagatagagaaagataggagataggagaaaggagaaagaggatgtggtattggatagaagggtaatgaggcacctcatcccctctttgggatgggcatgggtgaacacagcccaggcttttgttctggggctggaagagacaggcagaagggtctcagctgagcatcacatgaaagggctctgggggattggggcctcgtgacaggagcaaggcgggtggggtggggatggtgagagggtctggaatgtcccgtgctgctctgaggagggaggattgggagtggagaaagaatggggcatcttatgattctcttgttcttgtggtgaggtattcagtgggataattctagatcctcccccaagagaatcaaccaggtttctggtacatgttagagatggagtgaggatagtctgtgatgtgcagaaatatctacattgtaccccagtgccccctttctctagatccctggtctcacagacttcttggaacttctccttgatctgacttccctcattcatggtgtcatttcaagtcttattcttttactatgttcgttattgtattctggaaatatcctgttcatatgtgtccacccaaggctcttaatatgttgtgcttactttttggatccagatttttaaaatcataagaagacatttttatatagttcatgaaattttgcatggactgagtttgataattttgtttagtgtgaattaacattgtgtttatttaagaaaaaaaaatatttttttacagaaacctactgaatttgtagggttttaaaataacatgatgtctgggatttgcttttgaatgcttcagccaaaaaacaaacgaacaacaaaaataaaggatagataaagcaaatgtgacaaaatgctgatagttgttggaccttggggagacacatgcagagccatcacatcactttttttcagacatctttcttggtcagttataatcattttgtttgtccccactcccaatttctacttgcctctagtccatcctcctcactgcttgccaaagtgatccttctaaaacacaaatctgatcatattcaaaaagcttttgaagggtaagttttatggtatatgccatatatcagtacaacaaacaaaatcgtctgaggtgccgttgcctacaggataaagtccaaactcctttgcctggcactccaagcccccactctatcttcttggcctcatctctcatgatgtacatcagccacattgctagtgtctgctcatggccttctgcctagaatgctttatgccccagccaactatttactgtcttcttcagtcgaccagagtgcaatttacctgtttaaaatctatcattttgttatacattgtgcatgtctattatggctcatattaagcaatgccttggattatagtaatttatgtatatgtctatttcatatactttaacctgaaccccttcagaaccatttctttttcatttcttaagttctttgcacctagcccagtgcctggtacgtcgtgggtattcagtagattaaaatgcactttaaggaacttcccttgttgtccatcaagtggctaaggctctgtgctcccaatgcaggggaccagggttcaatctcaggtcagggaactagatcccacaggtcacaactaagagtttgcaagccacaactacctgacctcacatgccacaactaatcgaagatccctcgtgctgcaactaagtcctagtgcagttaaatatatttttttaatgcactttgaatgtgagaatgaatgatgtgtcacagacactgttgtcccctgagaagggagtgagtaatgatttgagggccctcatagtatatcttcctttttaggacccagagacagggactggcttggtgtctcaaggcagctcagaattaaagcatggaacagacagctgtatccagagtggacagaaagccaggggcctgactgctggagaggtaggaacttggcaatttccagggaggatatggtggaaatgggtggggaggggaacggggttgaatgtacttaggaagatagggaaggaaaaggcatacagggaggagaagccaaggagctaattaatgcagctgcccttttcaggtggccacatatccctgaaggtcagcaatgatgggcctacactgattggggcaaatgcttccttctctattgccttgcactttcctaaaagccaaaaggtgctgccagatgggcaggtcatctgggccaacaacaccatcatcaatggtgagtacctctccgcctccttcccaaggtccagaatccctggtatccccaatgagctcaaggaatcctcctcctctttttttttttttttttttacaaattatatatgtaacacatattcactgcagaaaaattagaaaacacagataaaccaaaaagaaaaaaaattatagttccccaaatggggcacagaagacccagtggacatagaagttggatagacttggatttaaactggttaccagtatgtgaccctggacaagtcactgaattgttttgttcttccattcccttatctatagaatggggatgataacactttaaaaggttcttgtaaggattaaaatgtgataatatataaagattttagcataatgcctgccctgtgctgtgcttagtaccttagtttagacgctttgcaaccccatggactgtagcccaccaggctcctctgtccatgtggattctgcaggcaagaatactggagtgggtcaccatgcactcctccaggggatcttcccaactcagggatcgaacccaggtcctagcctacagtattaattgatgctgttatttttacttttatcccactagctagagcacatcatcctagacattttgatacatggcctaccaatttgtgtccagtgtaagaatatacatgtgtgtgctcagtggctcagtcgtgtctgactctttgcaaccccatggactgtagcccgcgaaagctcctctgcccatgggattgcccagccaagaatactggagcaggttgccatttcttcctccaggggatctttcaacacagggattgaatccttgtctcctgtgtttcctgcattggcaggtgtattctttaccactgagccacctgggaaaccccttaagtatatacacataaatcttttatagtttccattctcccttctaccactccaaataggttataccaaggagaatgtattttggtagctaggcagtattcctggagcccctctctgggagtcatgttaaaggttttggtgtacagtgaggaatgccagggattgagggagacttgctgtcttcttttcagggagccaggtgtggggaggacagctggtatatccccaagaacctgatgatacctgcatcttccccgatggggagccctgcccttctggccctctatctcagaaaagatgctttgtttatgtctggaagacctggggtaagagtttcccttctctggcctgtcattcacacttaaattcacttcttcctacctgatcccctttcttttggtctcatccttaaattctgtgagtttccctaatcttcacttcccccatgactccttcctcttccacagcacctagtcaactctattatacttctttctgggagccctgctccaattatagtcccatcccatggaccctctcataaggacttttttcctgcccaacatatgcaagcttaaactctctgaaataaccatccttgatacatctcctgaccttccttctctggttccatctctaaccctgccccagtctcctttgaccagtaacccccttccctactcttctttccaaaaacctcagaccaatactggcaagttctggggggcccagtgtctggactgagcatcgggacagacaaggcaatgctgggcacatataacatggaagtgactgtctaccaccgccgggggtcccagagctatgtgcccctcgctcactccagttcagccttcaccattactggtaaggactgaggaggggacaaggccagttgcagggcaggagaaggtggggaggctgggctggacaggaaaggggaaagaggaaatggtgtgtaaccttacaggggcagaaccaggaagatgtgggcagagggatgtggggcttggagcccgtgaagggccaggcagcttgggttggttgaaaaatatggctgtgaaagaagaagctgacagaaagaagaacttatggttctcactttctctgactccaatcccagaccaggtgcccttctctgtgagtgtgtctcagctgcaggccttggatggaaggaacaagcgcttcctgagaaagcagcctctgacctttgccctccagctccatgatcccagtggctatttggctggggctgacctttcctacacctgggactttggtgacagtacagggaccctgatctctcgggcactcacggtcactcacacttacctagagtctggcccagtcactgcacaggtggtgctgcaggctgccattcctctcacctcctgtggctcctctccagttccaggcactacagataggcatgtgacaactgcagaggctcctggaaccacagctggccaagtgcctactacagaagtcatgggcaccacacctggccaggtgccaactgcagaggcccctggcaccacagttgggtgggtgccaaccacagaggatgtaggtaccacacctgagcaggtggcaacctccaaagtcttaagtacaacaccagtggagatgccaactgcaaaagctacaggtaggacacctgaagtgtcaactacagagccctctggaaccacagttacacagggaacaactccagagctggtggagaccacagctggagaggtgtccactcctgagcctgcgggttcaaatactagctcattcatgcctacagaaggtactgcaggtaagggggccaccatgaatgagttcatagaggtggggcatttgtcacagctctgaagacctgaaagaattgctcaggacccagatgttactcaatccttagcttagcagtggagtcccctcagaatcttcactggttttaaaaccccctaagtccctcttaatggcacagaatagatccagagttcaggaaaccagggtcttctcctaggccaggggtagagagcttattctctcttcctgaagagaagttcaggaagcagtgtgtgatcatttggtggtggtgctcagtcatgtctgactctttgtgacctcatggactatggcccaccaggctcctctgtccatagaattctccaggcaagaacactggagtgggtggccatttccttctccaggggattttccctgcccagggattaaacccgaattggcaggtggattctttacccgagccacctagaaagtcccatgtgatcattagataatacttatacctcattttctgattaagtgtaaacacagaaatctttctgacaccacttcccacccctggattcccatcccaaagtaggtttacctggaattgtggtaggaatactaaaaagggagaagtgagatagtgacactatgacttaacacatgtcaaatgtctgacccaggacctggcacagtgtagggtgtgataaacatttgggatgtctaaaattctgactctaaccctgtgactctggggcagtcatttctcttgggcctttctttatcttaaaaaatgagagtttccagctcttgtctgattctaagcctggatccagtagctctgactctacctggaaaaatgcttgttgggcctgttttcaggttagtcatttgctttttgactttgcctctttaatcctctcctccaggctccctgagtcccctgccggatgacactgccaccttagtcctggagaagcgccaagcccccctggattgtgttctgtatcgctatggctccttttccctcaccctggacattgtccgtgagtcttgcctacattgtccgtaagctggtggagggaggcgtgtgctgcttagggttgcccagtggaagcacaccttggaaggaattactcacctggacaaggagaatacccagatcccaggggtttcatatgaaggcagaatgggattagggaggcagcccgaggaccttcctggccatgggccttgggggaggataagtagaggagtctcagacttaaaaaaatcttgcaactttgcagagggtattgagagtgctgagatcctacaggctgtgtcatccagtgaaggagatgcatttgagctgactgtgtcttgccaaggcgggtgagtgtcccacggttgccctgagaactcctggggtgactgctgtcctgttctctggtgtctagtgtcccttcccagattccctgacgtaagctgacatctctcccaggctacccaaggaagcctgcatggacatctcatcgccagggtgtcagctgcctgcccagcggctgtgtcagcctgtgccccccagcccagcctgccagctggttttgcaccaggtactgaagggtggctcagggacctactgcctcaatgtgtctttggctgatgccaatagcctggcgatggtcagcacccagcttgtcatgcctggtaggtagttggacaagaggtaggatgaagacacggggagatggtagaggttacctactagaggaagcagacactgaatgcagccgtatctgggattccacccatagggcaagaagcaggcctcaggcaggctcctctgttcgtgggcatcttgctggtgctaacagctttgttgcttgcatctctgatatacaggtgagatccccgccatcctgctcccactcctttaccccttattaccaccaccactcttcctcatgggaagaagaaaccaccaacccctttgggaaagtgtagagtccaagaaagagcccagacttggaagttcaacaggtctaggctgcagtcttgctggtgggaccctggggaagtccattaacccttctgagccactgaaaagtaggaaacataatacctgtcctgtggggctgttttcagggctctagacaatgtgagtaaaacacctggttctgaaacaaaagtggaataaatgatgatctcaatgactgttgttatgaataatatcaacagtggagaagaactcagtgaactgagttctccacctgccagaaaggcaaatccctaggcctggagggctgaggtcctcaaagcagggaagcctgtagggtgagagggaaatggtcagagcttaccataaacataagagaggataaaccctgttggtgagaagaggagggagccaggatcaagaccaagtcaacctgggttatggtttagtcttttttttttagagaagcacaaagaggttgccattgaccaccactaaccagtatccctgcttttctcccaatatcaggcgaagacttatgaagcaaggctcagcagtcccccttccccagctgccacacggtagaacccagtggctacgtctgccctgggtcttccgctcttgccccattggtgagagcaaacccctcctcagtggacagcaggtctgagtgctcttatgtgaagtcatgatttacccaggtggacagcaaggcctgtcttttctctggtcttccctcagagactaccattgcctgaaataaagactcagaacttg

SEQ ID NO: 6 - protein, 648 aa, Bos taurus
MDLVLRKYLLHVALMGVLAVGTTEGPRDRDWLGVSRQLRIKAWNRQLYPEWTESQGPDCWRGGHISLKVSNDGPTLIGANASFSIALHFPKSQKVLPDGQVIWANNTIINGSQVWGGQLVYPQEPDDTCIFPDGEPCPSGPLSQKRCFVYVWKTWDQYWQVLGGPVSGLSIGTDKAMLGTYNMEVTVYHRRGSQSYVPLAHSSSAFTITDQVPFSVSVSQLQALDGRNKRFLRKQPLTFALQLHDPSGYLAGADLSYTWDFGDSTGTLISRALTVTHTYLESGPVTAQVVLQAAIPLTSCGSSPVPGTTDRHVTTAEAPGTTAGQVPTTEVMGTTPGQVPTAEAPGTTVGWVPTTEDVGTTPEQVATSKVLSTTPVEMPTAKATGRTPEVSTTEPSGTTVTQGTTPELVETTAGEVSTPEPAGSNTSSFMPTEGTAGSLSPLPDDTATLVLEKRQAPLDCVLYRYGSFSLTLDIVQGIESAEILQAVSSSEGDAFELTVSCQGGLPKEACMDISSPGCQLPAQRLCQPVPPSPACQLVLHQVLKGGSGTYCLNVSLADANSLAMVSTQLVMPGQEAGLRQAPLFVGILLVLTALLLASLIYRRRLMKQGSAVPLPQLPHGRTQWLRLPWVFRSCPIGESKPLLSGQQV

SEQ ID NO: 7 - DNA, 294 bp, Artificial sequence
  Probe
gttgctggaaggaagaacaggatggatctggtgctgagaaaataccttctccatgtggctctgatgggtgttcttctggctgtagggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactg

SEQ ID NO: 8 - DNA, 294 bp, Artificial sequence
  Probe
gttgctggaaggaagaacaggatggatctggtgctgagaaaataccttctccatgtggctctgatgggtgttcttctggctgtaaggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactg

SEQ ID NO: 9 - DNA, 291 bp, Artificial sequence
  Probe
gttgctggaaggaagaacaggatggatctggtgctgagaaaataccttctccatgtggctctgatgggtgttctggctgtagggaccacagaaggtgagtgtgggatgttggacatgaacaagtgtgaatttggggttgcacacctgctctggtttttctctccctaaaatggaagatatcagtagtgcttcaggtgtctcccacccatttgatttagtgaggacatgggcaactgagctccctccccacatgaagatttgggtgcatgtgtgttcaggcacttgggactg

SEQ ID NO: 10 - DNA, 30 bp, Artificial sequence
  Primer
gttgctggaaggaagaacaggatggatctg

SEQ ID NO: 11 - DNA, 30 bp, Artificial sequence
  Primer
cagtcccaagtgcctgaacacacatgcacc